(19) United States
(12) Patent Application Publication
(10) Pub. No.: US 2008/0071343 A1
(43) Pub. Date: Mar. 20, 2008

(54) Multi-segmented graft deployment system

(76) Inventors: Kevin John Mayberry, Mission Viejo, CA (US); Trinh Van Pham, Westminster, CA (US); Brian Christopher Gray, Lake Forest, CA (US); Stefan Schreck, Fallbrook, CA (US)

Correspondence Address: KNOBBE MARTENS OLSON & BEAR LLP, 2040 MAIN STREET, FOURTEENTH FLOOR, IRVINE, CA, 92614, US

(21) Appl. No.: 11/522,292

(22) Filed: Sep. 15, 2006

(51) Int. Cl. A61F 2/06

(52) U.S. Cl. 623/1.11; 606/108

(57) ABSTRACT

A deployment catheter for deploying endoluminal vascular prosthesis that has at least a main graft portion and a first branch graft portion includes an elongate, flexible catheter body having a proximal end and a distal end and comprising an outer sheath and an inner core that is axially moveable with respect to the outer sheath. The catheter includes a main graft restraint that has a main graft release mechanism comprising a plurality of axially spaced restraint members. The catheter further includes a branch graft restraint comprising a branch graft release mechanism.

### BACKGROUND OF THE INVENTION

1. Field of the Invention

The present invention relates to endoluminal vascular prosthesis deployment catheters, and in particular, to a deployment catheter for self-expanding prostheses comprising a main graft portion and at least one branch graft portion.

2. Description of the Related Art

An abdominal aortic aneurysm is a sac caused by an abnormal dilation of the wall of the aorta, a major artery of the body, as it passes through the abdomen. The abdomen is that portion of the body which lies between the thorax and the pelvis. It contains a cavity, known as the abdominal cavity, separated by the diaphragm from the thoracic cavity and lined with a serous membrane, the peritoneum. The aorta is the main trunk, or artery, from which the systemic arterial system proceeds. It arises from the left ventricle of the heart, passes upward, bends over and passes down through the thorax and through the abdomen to about the level of the fourth lumbar vertebra, where it divides into the two common iliac arteries.

The aneurysm usually arises in the infrarenal portion of the diseased aorta, for example, below the kidneys. When left untreated, the aneurysm may eventually cause rupture of the sac with ensuing fatal hemorrhaging in a very short time. High mortality associated with the rupture led initially to transabdominal surgical repair of abdominal aortic aneurysms. Surgery involving the abdominal wall, however, is a major undertaking with associated high risks. There is considerable mortality and morbidity associated with this magnitude of surgical intervention, which in essence involves replacing the diseased and aneurysmal segment of blood vessel with a prosthetic device which typically is a synthetic tube, or graft, usually fabricated of Polyester, Urethane, DACRON™, TEFLON™, or other suitable material.

To perform the surgical procedure requires exposure of the aorta through an abdominal incision which can extend from the rib cage to the pubis. The aorta must be closed both above and below the aneurysm, so that the aneurysm can then be opened and the

FIG. 23 is an exploded view of the peelable sheath of FIG. 22.

FIG. 24 is a cross-sectional view of the sheath of FIGS. 22-23 showing the temporary stitching holding the sheath compressed.

FIG. 25 is a cross-sectional view of the peelable sheath of FIGS. 22-23 showing an alternative embodiment of the stitching holding the sheath compressed.

### DETAILED DESCRIPTION OF THE PREFERRED EMBODIMENTS

Described below are various embodiments of a delivery system for deploying a vascular graft. In certain embodiments, the delivery systems is configured to deliver a graft that includes a main or distal graft portion and at least one branch or proximal graft portion. In such embodiments, the distal or main graft portion can be maintained in compressed state while the proximal or branch segment can be positioned within a branch vessel while in a compressed state. The delivery system can also be configured to allow the distal or main graft portion to be deployed while the he proximal or branch segment remains the compressed state. Other embodiments of a graft deployment system will also be described below. As used herein, the relative terms “proximal” and “distal” shall be defined from the perspective of the delivery system. Thus, proximal refers to the direction of the control end of the delivery system and distal refers to the direction of the distal tip.

With reference to FIG. 1, there is illustrated a schematic representation of the abdominal part of the aorta and its principal branches. In particular, the abdominal aorta 30 is characterized by a right renal artery 2 and left renal artery 4. The large terminal branches of the aorta 30 are the right and left common iliac arteries 37 and 38. Additional vessels (e.g., second lumbar, testicular, inferior mesenteric, middle sacral) have been omitted from FIG. 1 for simplification. One embodiment of an expanded bifurcated endoluminal vascular prosthesis is shown spanning aneurysms 103, 104 and 105. The expanded bifurcated endoluminal vascular prosthesis 50 can comprise a main branch portion 52 for traversing the aorta, a first branch portion 54 for spanning an ipsilateral iliac and a second branch portion 52 for spanning a contralateral iliac.

As depicted in FIG. 2, the bifurcated prosthesis 50 can comprise a polymeric sleeve 68 and a tubular wire support 60. In the illustrated embodiment, the thrombus, or blood clot, and arteriosclerotic debris removed. Small arterial branches from the back wall of the aorta are tied off. The DACRON™ tube, or graft, of approximately the same size of the normal aorta is sutured in place, thereby replacing the aneurysm. Blood flow is then reestablished through the graft. It is necessary to move the intestines in order to get to the back wall of the abdomen prior to clamping off the aorta.

If the surgery is performed prior to rupturing of the abdominal aortic aneurysm, the survival rate of treated patients is markedly higher than if the surgery is performed after the aneurysm ruptures, although the mortality rate is still quite high. If the surgery is performed prior to the aneurysm rupturing, the mortality rate is typically slightly less than 10%. Conventional surgery performed after the rupture of the aneurysm is significantly higher, one study reporting a mortality rate of 66.5%. Although abdominal aortic aneurysms can be detected from routine examinations, the patient does not experience any pain from the condition. Thus, if the patient is not receiving routine examinations, it is possible that the aneurysm will progress to the rupture stage, wherein the mortality rates are significantly higher.

Disadvantages associated with the conventional, prior art surgery, in addition to the high mortality rate include the extended recovery period associated with such surgery; difficulties in suturing the graft, or tube, to the aorta; the loss of the existing aorta wall and thrombosis to support and reinforce the graft; the unsuitability of the surgery for many patients having abdominal aortic aneurysms; and the problems associated with performing the surgery on an emergency basis after the aneurysm has ruptured. A patient can expect to spend from one to two weeks in the hospital after the surgery, a major portion of which is spent in the intensive care unit, and a convalescence period at home from two to three months, particularly if the patient has other illnesses such as heart, lung, liver, and/or kidney disease, in which case the hospital stay is also lengthened. The graft must be secured, or sutured, to the remaining portion of the aorta, which may be difficult to perform because of the thrombosis present on the remaining portion of the aorta. Moreover, the remaining portion of the aorta wall is frequently friable, or easily crumbled.

Since many patients having abdominal aortic aneurysms have other chronic illnesses, such as heart, lung, liver, and/or kidney disease, coupled with the fact that many of these patients are older, the average age being approximately 67 years old, these patients are not ideal candidates for such major surgery.

More recently, a significantly less invasive clinical approach to aneurysm repair, known as endovascular grafting, has been developed. Parodi, et al. provide one of the first clinical descriptions of this therapy. Parodi, J. C., et al., “Transfemoral Intraluminal Graft Implantation for Abdominal Aortic Aneurysms,” 5 Annals of Vascular Surgery 491 (1991). Endovascular grafting involves the transluminal placement of a prosthetic arterial graft within the lumen of the artery.

Endoluminal repair or exclusion of aortic aneurysms has been performed for the past several years. The goal of endoluminal aortic aneurysm exclusion has been to correct this life threatening disease in a minimally invasive manner in order to effectuate a patient's quick and complete recovery. Various vascular grafts exist in the prior art that have been used to exclude aortic aneurysms. In general, transluminally implantable prostheses adapted for use in the abdominal aorta comprise a tubular wire cage surrounded by a tubular PTFE or Dacron sleeve. Both balloon expandable and self expandable support structures have been proposed. Endovascular grafts adapted to treat both straight segment and bifurcation aneurysms have also been designed.

Endoluminal implantation is an increasingly accepted technique for implanting vascular grafts. Typically, this procedure involves percutaneously inserting a vascular graft or prosthesis by using a delivery catheter. This process eliminates the need for major surgical intervention thereby decreasing the risks associated with vascular and arterial surgery. Various catheter delivery systems for prosthetic devices are described in the prior art.

For example, current delivery systems for a bifurcated stent graft system or a graft having at least one branch portion use two sheaths moving in opposing directions to deploy the distal segment of the graft before the proximal segment. The outer sheath is first retracted to deploy a portion of the mid-body and the contralateral limb. Then, the front sheath is advanced distally to deploy the distal end of the graft. See e.g., U.S. Pat. No. 6,660,030. While successful, it may be advantageous to limit the distal movement of the front sheath.

### SUMMARY OF THE INVENTION

Accordingly, one aspect of the present invention comprises a deployment catheter for deploying endoluminal vascular prosthesis that has at least a main graft portion and a first branch graft portion. The catheter includes an elongate, flexible catheter body having a proximal end and a distal end and comprising an outer sheath and an inner core that is axially moveable with respect to the outer sheath. The catheter can also include a main graft restraint that has a main graft release mechanism comprising a plurality of axially spaced restraint members. The catheter can further include a branch graft restraint comprising a branch graft release mechanism.

Another aspect of the present invention is a deployment catheter that comprises a flexible outer tubular member, having a proximal end and a distal end. An intermediate tubular member is slidably engaged with the outer tubular member and has a proximal end and a distal end. A central core is slidably engaged with the intermediate tubular member and having a proximal end and a distal end. A flexible, conical tip is mounted on the distal end of the central core. A main graft restraint is operatively engaged with the intermediate tubular member and the central core and configured such that proximal retraction of the intermediate tubular member relative to the central core will release the main graft restraint. Proximal retraction of the outer tubular member will release the first compressed branch graft portion.

Another aspect of the present invention is a deployment catheter that includes a flexible outer tubular member and a central core slidably engaged with the outer tubular member. A main graft portion is disposed on the central core and constrained in a compressed state by an internal restraint having a first release mechanism. A compressed branch graft portion has a second release mechanism. The compressed branch graft portion constrained by the flexible outer tubular member. The compressed branch graft portion is released by proximal traction of the outer tubular member. The first release mechanism is engaged by proximal traction.

Another aspect of the present invention comprises a method of deploying an endoluminal vascular prosthesis in a patients aortic artery in which A deployment catheter containing an endoluminal vascular prosthesis comprising a compressed main graft portion, a compressed ipsilateral branch portion and a compressed contralateral branch portion is advanced beyond a bifurcation in the aorta. An outer sheath of the deployment catheter is proximally retracted to expose the main graft portion and the contralateral branch portion of the prosthesis. A release member is retracted to release a plurality of release mechanisms axially spaced along the main graft to release the main graft portion.

### BRIEF DESCRIPTION OF THE DRAWINGS

FIG. 1 is a schematic representation of a bifurcated vascular prosthesis for use with the present invention, positioned at the bifurcation between the abdominal aorta and the right and left common iliac arteries.

FIG. 2 is an exploded view of a bifurcated graft for use with the present invention, showing a self-expanding wire cage separated from an outer polymeric sleeve.

FIG. 3 is a schematic representation of an embodiment of the deployment system of the present invention

FIG. 4 is a schematic representation of a restraining system for constraining a main graft portion of a vascular prosthesis for use the deployment system of FIG. 3.

FIG. 5 is a cross-sectional view of the restraining system of FIG. 4.

FIG. 6 is a schematic representation of an embodiment of the opposing connectors for use with the restraining system of FIG. 4.

FIG. 7 is a schematic representation of an alternative embodiment of the opposing connectors for use with the restraining system of FIG. 4.

FIG. 7A is an expanded view of an opposing connector of the restraining system of FIG. 7.

FIG. 8 is a schematic representation of the restraining system of FIG. 7 in a restraining position.

FIG. 9 is a schematic representation of the restraining system of FIG. 7 in a released configuration.

FIG. 10 is a cross-sectional view of an embodiment of the intermediate member of the deployment system of FIG. 3.

FIG. 11 is a cross-sectional view of an alternative embodiment of the intermediate member of the deployment system of FIG. 3.

FIG. 12 is a schematic representation of the deployment system of FIG. 3 being advanced through the abdominal aorta.

FIG. 13 is a schematic representation of the deployment system of FIG. 3 showing outer sheath retracted and the contralateral branch portion positioned in the contralateral iliac.

FIG. 14 is a schematic representation of the deployment system of FIG. 3 contralateral branch portion deployed in the contralateral iliac.

FIG. 15 is a schematic representation of the deployment system of FIG. 3 showing the bifurcated vascular prosthesis fully deployed in the abdominal aorta and iliac arteries.

FIG. 16 is a schematic representation of an alternative embodiment of the deployment system of the present invention.

FIG. 17 is a schematic representation of an alternative embodiment of the deployment system of the present invention.

FIG. 18A is a schematic representation of a restraint member for use with the deployment system of FIGS. 16-17.

FIG. 18B is a cross-sectional view of the restraint member of FIG. 18A.

FIG. 19A is a schematic representation of an alternative embodiment of a restraint member for use with the deployment system of FIGS. 16-17.

FIG. 19B is a cross-sectional view of the restraint member of FIG. 19A.

FIG. 19C is a cross-sectional view of the restraint member of FIG. 19A showing a release wire engaged with one of the grooves.

FIG. 20 is a schematic representation of an alternative embodiment of the deployment system of the present invention.

FIG. 21 is a schematic representation of an alternative embodiment of the deployment system of the present invention.

FIG. 22 is a schematic representation of an embodiment of a peelable sheath.

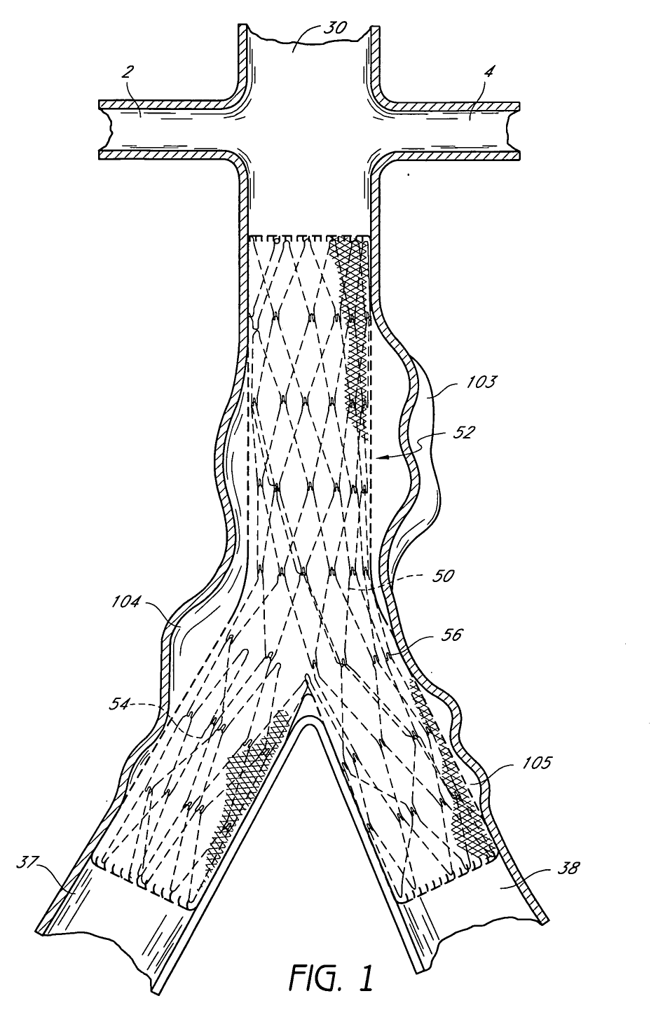

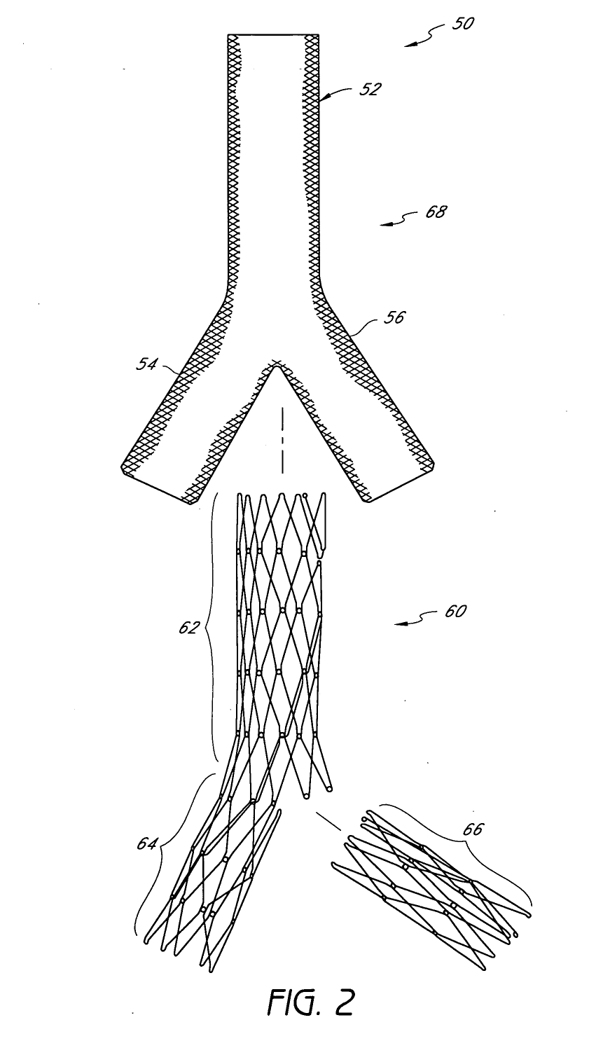

polymeric sleeve 60 can be situated concentrically outside of the tubular wire support 60. However, other embodiments may include a sleeve situated instead concentrically inside the wire support or on both of the inside and the outside of the wire support. Alternatively, the wire support may be embedded within a polymeric matrix or layer which makes up the sleeve. The sleeve 68 may be attached to the wire support 60 by any of a variety of suitable manners known to those skilled in the art.

The tubular wire support 60 can comprise a main branch portion 62 for traversing the aorta, a first branch portion 64 for spanning an ipsilateral iliac and a second branch portion 66 for spanning a contralateral iliac. The main branch portion 62 and first ipsilateral branch portion 64 can be formed from a continuous single length of wire having a proximal end, a distal end and a central lumen extending therebetween. Alternatively, the first ipsilateral branch portion 64 may be formed of one or more lengths of wire pivotably connected to the proximal end of the main branch portion 62. A second, contralateral branch component 66 may be formed of one or more lengths of wire pivotably connected to the proximal end of the main branch portion 62. Each of the iliac branch components has a proximal end, a distal end and a central lumen extending therethrough. Construction of the graft from a three part cage conveniently facilitates the use of different gauge wire in the different components (e.g. 0.014″ diameter main trunk and 0.012″ diameter branch components).

In general, each of the components of the bifurcated endoluminal vascular prosthesis 50 may be varied considerably in diameter, length, and expansion coefficient, depending upon the intended application. For implantation within a typical adult, the main branch portion 52 will have a length within the range of from about 5 cm to about 12 cm, and, typically within the range of from about 9 cm to about 10 cm. The unconstrained outside expanded diameter of the main branch portion 52 will typically be within the range of from about 20 mm to about 40 mm. The unconstrained expanded outside diameter of the main branch portion 52 can be constant or substantially constant throughout the length, or can be tapered from a relatively larger diameter at the distal end to a relatively smaller diameter at the bifurcation. In general, the diameter of the proximal end of the main branch portion will be on the order of no more than about 95% and, preferably, no more than about 85% of the diameter of the distal end of the main branch portion. The iliac branch portions 54,56 will typically be bilaterally symmetrical, having a length within the range of from about 1 cm to about 6.5 cm, and a diameter within the range of from about 10 mm to about 20 mm.

The collapsed prosthesis for use in accordance with the present invention has a diameter in the range of about 2 mm to about 10 mm. Preferably, the maximum diameter of the collapsed prosthesis is in the range of about 3 mm to 6 mm (12 to 18 French). Some embodiments of the delivery catheter including the prosthesis will be in the range of from 18 to 20 or 21 French; other embodiments will be as low as 19 F, 16 F, 14 F, or smaller. After deployment, the expanded endoluminal vascular prosthesis may radially self-expand to a diameter anywhere in the range of about 20 to 40 mm.

Although certain prosthesis configurations are disclosed herein, these are only examples of prostheses which are deployable using the embodiments of a deployment catheter described herein. In other embodiments, the deployment catheter described below may be used to deliver and deploy other types of self expandable bifurcated or multi-segmented prosthesis having a main graft portion and at least one branch graft portion, as will be apparent to those of skill in the art in view of the disclosure herein. In other embodiments, certain features and aspects of the deployment catheter can be used to deploy a graft without a branch graft portion, a graft with only one branch portion and/or a graft with more than one graft portions. Further details and additional embodiments of the prosthesis described above can be found in U.S. Pat. Nos. 6,007,296, 6,187,036, 6,197,049, 6,500,202, 6,660,030, 6,654,475, 6,261,316 and 6,663,665 and U.S. Patent Publication No. 2004/0167618, the entirety of these patents and patents applications hereby incorporated by reference herein.

It should also be appreciated that, although the illustrated embodiments are described in the context of a bifurcated graft configured for the abdominal aorta, certain features and aspects of the deployment systems and methods described herein can be used in other portions of the vascular system. For example, it is anticipated that certain features and aspects of the systems and methods described herein can be adapted for use in the thoracic aorta. It is also anticipated that certain features and aspects of the system described herein may be adapted to deliver a single straight graft segment to the thoracic aorta.

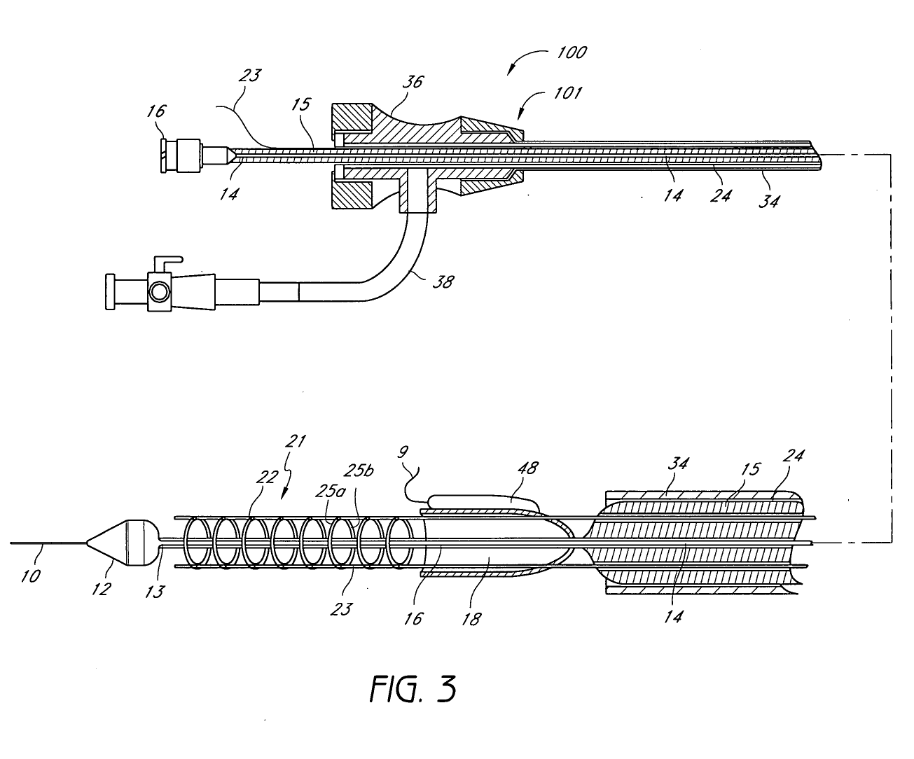

FIG. 3 is a cross-sectional side view of one embodiment of a delivery system 100 for deploying a multi-segment vascular prosthesis, such as the prosthesis 50 described above. In this embodiment, the system 100 comprises an elongate flexible multi-component tubular body 101. The tubular body 101 and other components of this system may be manufactured in accordance with any of a variety of techniques which are well known in the art of catheter manufacturing, such as by braiding and/or extrusion. Suitable extrudable materials include high density polyethylene, medium density polyethylene and other polyethylene blends, nylon, PEBAX, and others well known in the art. Reinforced tubular bodies may be produced by including a braided or coiled layer in or on the wall. The braided or coiled layer may comprise any of a variety of materials such as stainless steel, nitinol, composite fibers and others known in the art. Suitable dimensions can be readily selected taking into account the natural anatomical dimensions in the iliacs and aorta, together with the dimensions of the desired percutaneous access site. In certain embodiments, the tubular body 101 may have an overall length of between about 90-110 cm so that the delivery system can deploy an abdominal aortic aneurysm graft through an access in the femoral artery.

The multi-component tubular body 101 comprises an inner or central core 24, and an outer sheath 34. In certain embodiments, the central core 24 may be axially movably positioned within the outer sheath 34. In certain embodiments, the central core 24 may be axially movably positioned within but rotationally locked to the outer sheath 34. In this manner, the rotational orientation of the central core 24 can remain fixed with respect to the rotational orientation of the outer sheath 34.

Rotational engagement can be accomplished in any of a variety of ways, normally involving complementary surface structures such as keys or splines on the associated components. For example, the central core 24 can be provided with a radially outwardly extending projection, along a portion or all of its axial length. This projection can be slidably received within a radially outwardly extending slot on the interior surface of the outer sheath 34, or a component secured thereto. Alternatively, a radially inwardly extending projection on the outer sheath 34 or an associated component can be received with an axially extending recess on the outer surface of the central core 24. Alternatively, any of a variety of non-round configurations for the central core 24, such as elliptical, oval, triangular, square, polygonal, and the like, can be slidably received within a complementary-shaped lumen in the outer sheath 34.

In the illustrated embodiment, the cross section of the central core 24 deviates from circular by the provision of one or two opposing flat sides extending axially along its length. A corresponding aperture can be provided in a manifold 36 positioned at the proximal end of the central core 24. Thus, rotation of the outer sheath 34 in this embodiment will cause a similar rotation of the central core 24. Similarly, the central core 24 can be provided with one or two opposing flat surfaces to be slidably received through a complementary aperture in a rotational lock on a manifold 36 connected to the proximal end of the outer sheath 34. The resulting assembly can enable rotation of the manifold 36 to cause a commensurate rotation of the outer sheath 34 and central core 24. Specific dimensions and design details of the rotational lock disclosed herein will be readily apparent to those of skill in the art in view of the disclosure herein. In alternative embodiments, for example wherein the delivery system is configured to deliver a single, straight graft segment, a rotational lock between the central core 24 and the outer sheath 34 may not be provided.

In the illustrated embodiment, the tubular outer sheath 34 can be configured to cover the entire multi-segmented graft and connect or mate with a proximal end of a distal cap 12 mounted on the distal end 13 of the central core 24 in order to provide a smooth, continuous exterior for advancing the system 100 through the patient's vasculature. As mentioned above, the tubular sheath 34 can further include at its proximal end a manifold 36. The manifold 36 can have a hemostatic valve 38, which can provide an access port for the infusion of drugs or contrast media as will be understood by those of skill in the art. In one embodiment, the outer sheath 34 may comprise extruded PTFE, having an outside diameter of between 22 French and 18 French and in one embodiment 20 French. The outer sheath 34 can have an axial length of between about 90-110 cm, which in certain embodiments is sufficient to cover the vascular graft and connect with the distal tip 12 in a deployment configuration.

The central core 24 can comprise an elongate tubular body having a central lumen 14 adapted to axially slidably or track over a guide wire 10. In certain embodiments, the central core 24 may have a varying cross-sectional diameter such that a proximal region 15 of the central core 24 has a cross-sectional diameter sized to be slidably insertable in the lumen of the outer sheath 34 which necks down to a distal region 16 which has a cross-sectional diameter only slightly larger than the central lumen 14. In such embodiments, the smaller diameter of the distal region 16 provides an annular space between the central core 24 and the outer sheath 34 wherein a constrained vascular graft may be positioned during delivery. Accordingly, the distal region 16 preferably had a length greater than the maximum length of a vascular graft for use with the delivery system 100. In certain embodiments, the central core 24 can comprise a polyethelene or PTFE extrusion with a proximal region 15 has an outside diameter of about 0.220 inches tapering down to a small diameter, thin-walled tube in the distal region 16 and having at least one central lumen 14 extending axially through both the proximal 15 and distal 16 regions of the tubular body. The inner and/or outer surfaces of the proximal region 15 can be provided with a lubricious coating such as paralene, silicone, PTFE or others well known in the art to facilitate axial movement of the central core 24 within the outer sheath 34.

With reference now to FIGS. 3-5 and the distal region 16 of the delivery system 100, a restraint system 21 for compressing the main body portion 52 of the multi-segmented graft 50 can be coupled to the central core 24. As will be explained below, in the illustrated embodiment, the restraint system 21 comprises an external restraint device for compressing the main graft portion. The restraint system 21 can also have a pullback release mechanism for actuating the restraint device. For example, in the illustrated embodiment, the restraint system 21 comprises a restraining wire 22 having a plurality of opposing connectors 25a, b configured to surround and constrain the main graft portion 52 and a release wire 23 configured to alternately connect and release the opposing connectors 25a, 25 extending from the distal end of the intermediate member 24.

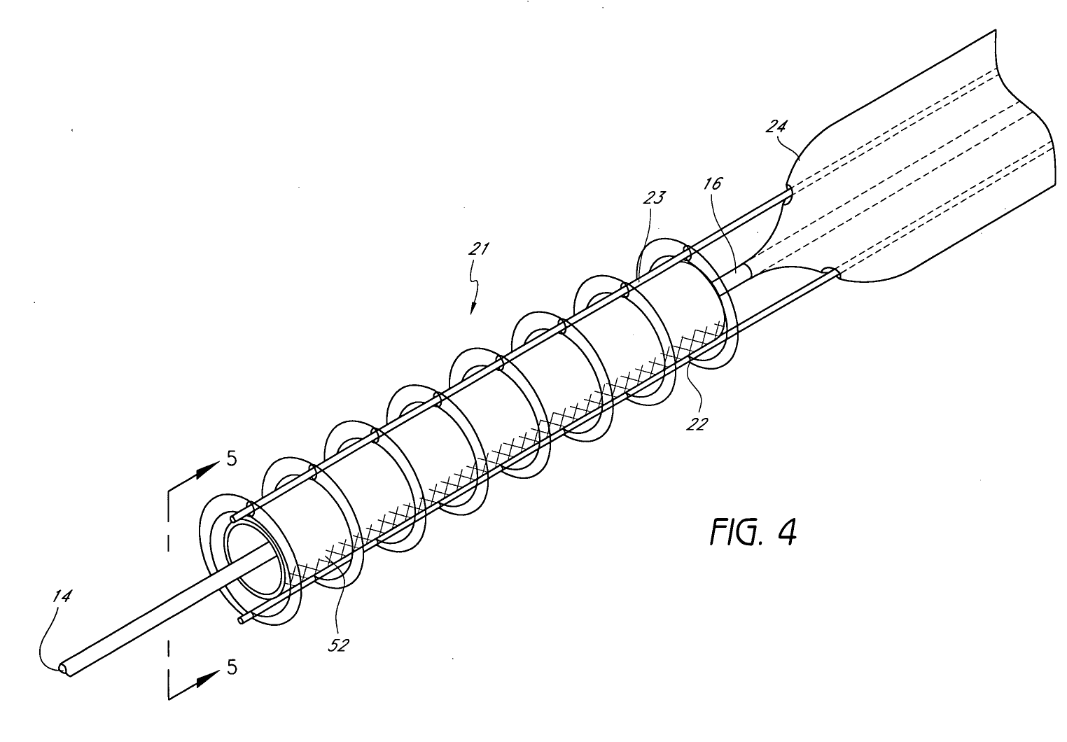

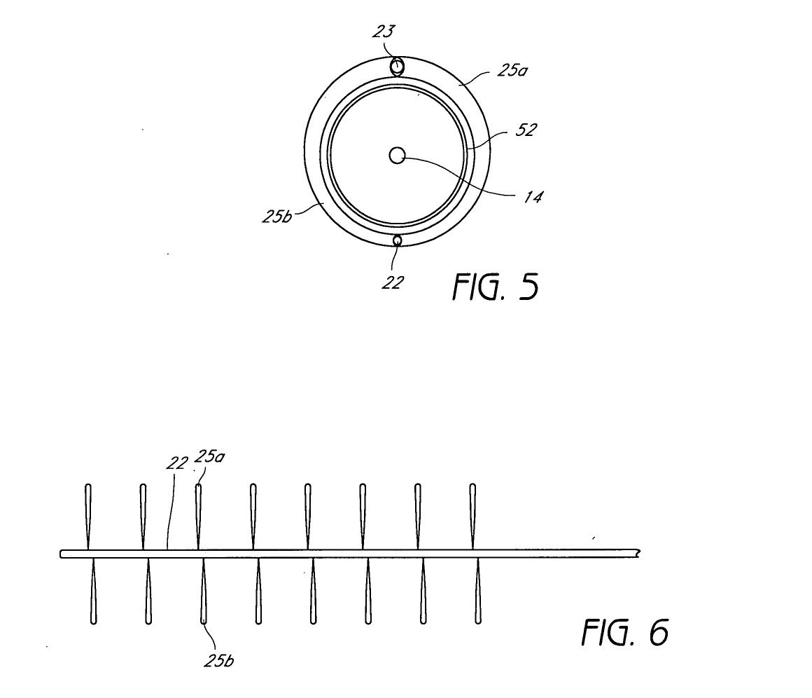

FIGS. 4-6 illustrate the restraining wire 22 and the plurality of pairs of opposing connectors mechanisms 25a-b spaced apart along the distal end of the restraining wire 22. In one embodiment, as depicted in FIG. 6, the pairs of connectors can comprise pairs of loops of wire or thread 25a-b. The pairs of loops 25a-b can be spaced apart along a length of the restraining wire 22 corresponding to the length of the main graft portion 52 such that the main graft portion 52 may be completely constrained by the paired loops 25a-b (see e.g., FIGS. 4 and 5). In the illustrated embodiment, the restraining wire 22 comprises eight pairs of loops 25a-b spaced apart along the distal end of the restraining wire 22. However, in modified embodiments, more or fewer pairs of loops may be provided depending upon the length of the main graft portion 52 to be constrained and the desired spacing between the pairs of loops 25a-b. The restraint wire and the loops 25a-b can be formed from the same or similar material. For example, in one embodiment, restraint wire and the loops 25a-b can comprise polypropylenes, plastics, polymers, fibers or any suitable materials known in the art.

In use, the release wire 23 can be configured to extend through the each pair of loops 25a-b to hold the opposing loops 25a, 25b together, as shown in FIGS. 4-5. The main portion of the graft 52 can be mounted over the distal region 16 of the central core 24 and positioned on the restraining wire 22 such that when the paired loops 25a-b are connected by the release wire 23, the main graft portion 52 is restrained in a compressed configuration by the paired loops 25a-b. Thus, when the release wire 23 is pulled proximally with respect to the restraining wire 22, the loops 25a-b are released and the main graft portion 52 may self-expand to its unconstrained diameter. The release wire 23 can be formed from the same or similar material as the restraining wire 22. For example, in one embodiment, release wire 23 can comprise any suitable plastic, polymer, metal, Nitinol fiber or any other suitable material known in the art.

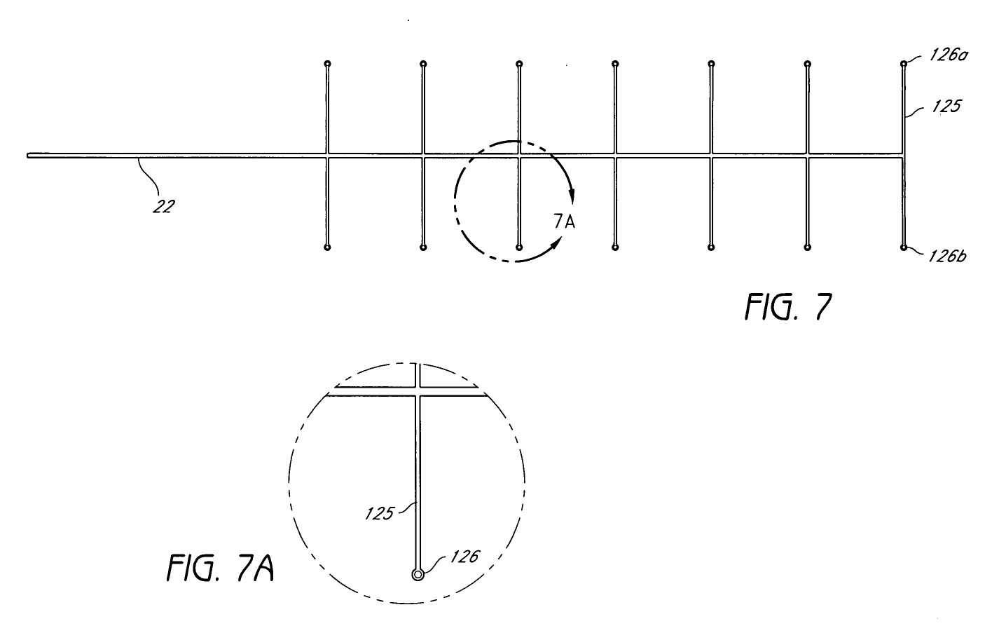

In a modified embodiment depicted in FIGS. 7-7A, the opposing connectors may comprise a plurality of discontinuous or open rings 125 connected in the middle to the restraining wire 22. The rings 125 can include eyelets 126a-b mounted on the ends of the ring 125 such that the ring 125 may be cinched together by aligning the eyelets 126a-b and weaving the release wire 23 through both eyelets 126a-b. The rings 125 can be spaced apart along a length of the restraining wire 22 corresponding to the length of the main graft portion 52 such that the main graft portion 52 can be completely constrained by the rings 125. In the illustrated embodiment, the restraining wire 22 comprises seven rings 125 spaced apart along the distal end of the restraining wire 22. However, in alternative embodiments, other numbers of rings may be provided depending upon the length of the main graft portion 52 to be constrained and the desired spacing of the rings 125.

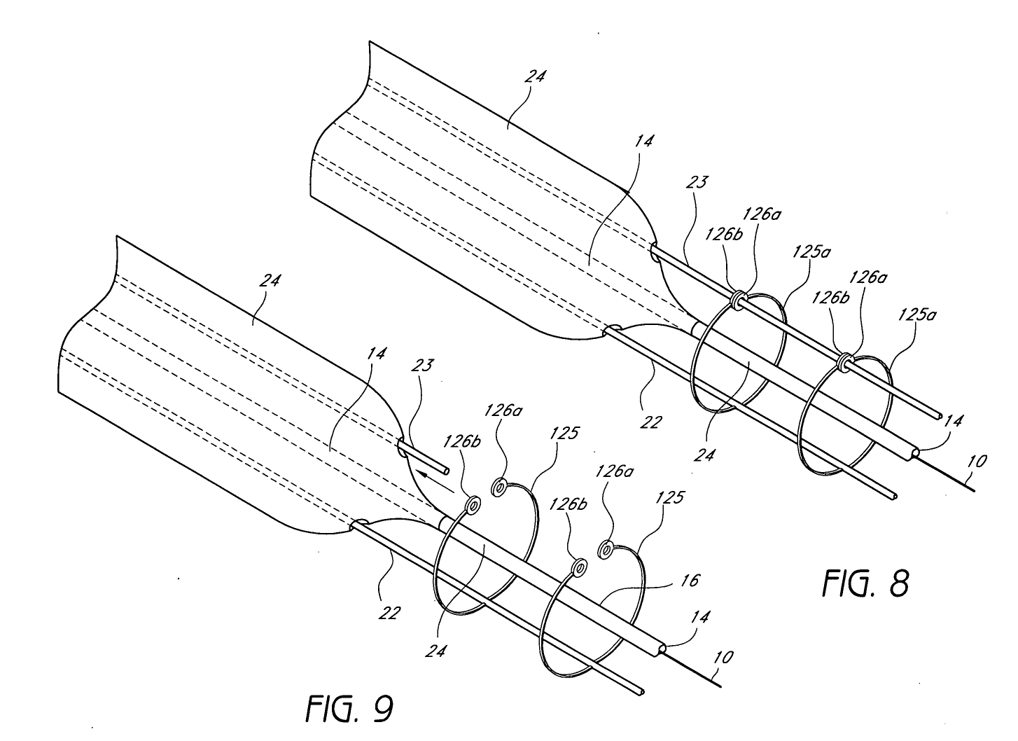

In use, as depicted in FIGS. 8-9, the release wire 23 is configured to be extended through the eyelets 126a-b of each ring to hold the rings 125 tight. The main portion of the graft (not shown in FIGS. 8-9) can be mounted over the distal region 16 central core 24 and positioned on the restraining wire 22 such that when the eyelets 126a-b of each ring 125 are joined, the main graft portion is restrained by the rings 125. Thus, when the release wire 23 is pulled proximally with respect to the restraining wire 22, the eyelets 126a-b are disconnected and the main graft portion can be released and self-expand to its unconstrained diameter. In certain embodiments, the restraining wire 22 and loops may also be pulled proximally once the main graft portion has been released in order to retract the restaining wire 22 and rings 125 into the outer sheath 34 before withdrawing the delivery system from the patient's vasculature.

An advantage of the embodiments described with reference to FIGS. 4-9 is that a distal portion of the main graft portion can be expanded first as the release wire 23 is proximally retracted. In such a manner, the main graft portion can be expanded from the distal portion to the proximal portion. However, those of skill in the art will recognize that the above described embodiments can be modified such that a proximal or middle portion of the main graft portion is released first. In such an embodiment, the system 100 can be provided with a plurality of release wires. For example, a first release wire can be threaded through a first group of eyelets or loops associated with a proximal portion of the main graft portion. A second release wire can be threaded through a second group of eyelets or loops associated with a distal portion of the main graft portion. In this manner, proximal retraction of the first release wire will allow the proximal portion of the main graft portion to expand while the distal portion of the main graft remains constrained in a compressed configuration. In modified embodiment, the structure described above can be used to release the main graft portion in discrete sections.

In an alternative embodiment, the release system described in FIGS. 8-9 may alternatively be used to deliver a single straight vascular graft to an aneurysm located in a sharply curved blood vessel, such as the thoracic aorta. In such embodiments, the looped release system may be advantageous in that it provides increased flexibility in maneuvering the compressed graft through a tortuous path and thus can reduce the risk of trauma or puncturing the blood vessel walls as the delivery system is advanced to the aneurysm. For example, the loops provide a series of discrete tension points spaced apart along the length of the graft segment for restraining the graft as opposed to a sheath which provides a stiff restraint layer extending the entire length of the graft. Accordingly, the loops with their spaced apart tension points allow the constrained graft segment to bend more easily, providing increased flexibility to the delivery system as it is advanced through a patient's blood vessel and particularly allowing for greater flexibility along the sharply curved sections of the blood vessel.

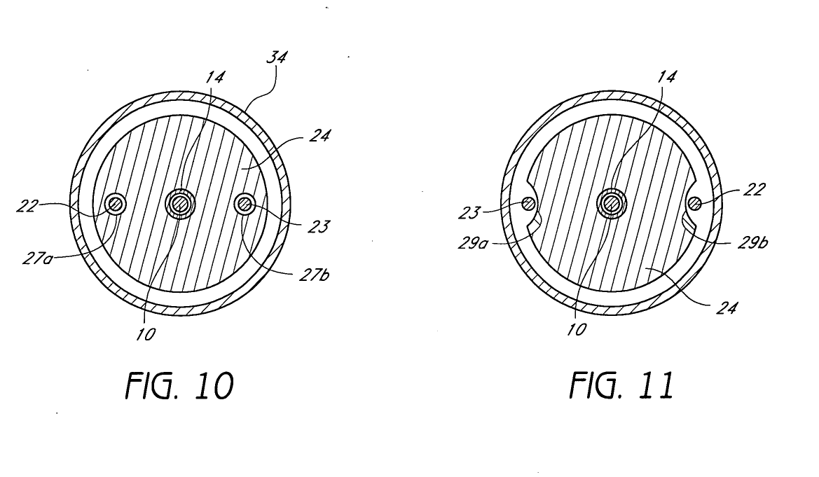

With reference now to FIG. 10, in certain embodiments, the proximal region 15 of the central core 24 can comprise an extruded tubular member having a central lumen 14 for tracking over the guide wire 10 and a pair of lumens 27a-b extending axially along the length of the central core 24 on opposing sides of the central core 24. The restraining wire 22 can extend through one lumen 27a in the central core 24. The proximal end of the restraining wire 22 can be fixedly secured to the proximal end of the central core 24 while the distal end of the restraining wire 22 can extend freely from the distal end of the proximal region 15 of the central core 24. The restraining wire 22 has a length such that the distal end of the restraining wire 22 may extend along the length of the distal region 16 of the central core 24. In certain embodiments, the restraining wire 22 extends along the distal region 16 of the central core 24 to the distal tip 12 of the central core 24. Alternatively, the restraining wire 22 may extend a suitable length to fully constrain a vascular graft positioned on the distal region 15 of the central core 24. In a modified embodiment, the restraining wire 22 may extend freely from the proximal end of the core member 24 such that the restraining wire 22 may be pulled proximally once the main graft portion has been released in order to retract the distal end of restraining wire 22 and the connectors 25a-b into the outer sheath 34 before withdrawing the delivery system from the patient's vasculature.

The release wire 23 can also extend from the distal end of the proximal region 15 of the central core 24 and parallel to the restraining wire 22. The release wire 23 can extend through the other lumen 27b in the central core 24. In a modified embodiment, the release wire 23 can extend through the same lumen as the restraint wire 22. The release wire 23 can be configured to be axially slidable in the lumen 27b and have a length such that the proximal and distal ends of release wire 23 extend from each end of the lumen 27a in the proximal region 15 of the central core 24. Preferably, the release wire 23 has a length such that when the release wire 23 is engaged with the opposing connectors 25a-b on the restraining wire 22, the proximal end of the release wire 23 extends from the proximal end of the central core 24 such that it may be gripped and pulled proximally by an operator to release the opposing connectors 25a-b.

In a modified embodiment depicted in FIG. 11, the proximal region 15 of the central core 24 can comprise an extruded tubular member having a central core lumen 14 for tracking over the guide wire 10 and a pair of grooves 29 a-b extending axially along the length of the central core 24 on opposing sides of the central core 24. The release wire 23 and the restraint wire 22 can be configured to extend axially along these grooves 29a-b in the annular space formed between the central core 24 and the outer sheath 34. As described above, the restraining wire 22 can be fixedly attached to the connector 26 at the proximal of the central core 24, while the release wire 23 may further extend through an aperture in the proximal end of the central core 24. The release wire 23 may then be pulled proximally through the groove 29b in the central core 24 with respect to the attached restraint wire 22 to release the main graft portion.

As depicted in FIG. 3, a smaller diameter distal region 16 of the central core 14 extends from the proximal region 15 of the central core 24 The distal region 16 comprises a lumen 14 for advancing the delivery system over a guidewire 10 to the aneurysm. A tapered distal tip 12 can be secured to the distal end 13 of the distal region 16 to facilitate insertion and atraumatic navigation of the delivery system through the patient's vasculature. The proximal end of distal tip 12 is preferably directly or indirectly connected to the distal end 13 such as by a friction fit and/or adhesive bonding. The distal tip 12 can have a maximum outer diameter such that the tip may securely fit into the distal opening of the outer sheath 34 during insertion into and navigation of the vasculature. The length and taper of the distal tip 12 can be varied depending upon the desired trackability and flexibility characteristics.

The distal region 16 is preferably a thin-walled tube designed to track over a guidewire, such as a standard 0.035 guidewire. The distal region 16 preferably has as small an outside diameter as possible to minimize the over all outside diameter of the delivery system and to provide sufficient annular space between the distal region 16 and the outer sheath 34 for housing the compressed vascular graft, while still providing sufficient column strength to support a compressed vascular stent housed on thereon. In use, a vascular graft may be slidably positioned over the distal region 16 of the central core 24. The restraining wire 22 may then be axially advanced until the restraining wire 22 is located alongside the distal, main branch segment of the graft such that the wire loops 25a-b may be wrapped around the main branch segment. The release wire 23 may then be advanced and woven through each pair of loops 25a-b to compress and constrain the main segment of the graft.

As shown in FIG. 3, an open ended tubular sheath 18 can be secured to the distal region 16 of the central core 24 for holding a proximal, branch graft portion 54 of the vascular graft in a compressed state. The sheath 18 is preferably coupled to the distal region 16 of the central core 24 such that the main graft segment 52 can be positioned distal to the sheath 18 and the sheath 18 can therefore restrain the proximal, ipsilateral branch portion 54 of the graft 50 in a compressed configuration. In one embodiment, the sheath 18 comprises a thin walled PTFE extrusion having an outer diameter of about 0.215″ and an axial length of about 7.5 cm. The proximal end of the sheath 18 may be necked down such as by heat shrinking to secure the tubular sheath 18 to the central core 24 while leaving the distal end of the sheath 18 open. In certain embodiments, the restraining wire 22 and release wire 23 may extend through the sheath 18, alternatively, the restraining wire 22 and release wire 23 may extend between the sheath 18 along side the sheath 18. Proximal withdrawal of the central core 24 will in turn proximally retract the sheath 18, thereby deploying the ipsilateral branch graft portion 54.

As described above, in certain embodiments, a second, contralateral branch graft portion 56 can also extend from the proximal end of main graft portion 52. In certain embodiments, the contralateral branch portion 56 of the vascular graft 50 can be constrained within a second sheath 48 that can be secured to a contralateral guide wire 9 and positioned alongside the constrained ipsilateral branch portion 54 in the annular space between the sheath 18 and the outer sheath 34. The second sheath 48 for constraining the contralateral branch portion can have a significantly smaller cross-section than the tubular sheath 18 due to the presence of the central core 24 extending through the tubular sheath 18.

The second sheath 48 can also be secured at its proximal end to a distal end of the contralateral guide wire 9 though any of a variety of securing techniques, such as heat shrinking, adhesives, mechanical interfit and the like. In one embodiment, the guidewire 9 is provided with a knot or other diameter enlarging structure to provide an interference fit with the proximal end of the second tubular sheath 48 and the proximal end of the second tubular sheath 48 can be heat shrunk and/or bonded in the area of the knot to provide a secure connection. Any of a variety of other techniques for providing a secure connection between the contralateral guidewire 9 and tubular sheath 48 can readily be used in the context of the present invention as will be apparent to those of skill in the art in view of the disclosure herein. The contralateral guidewire 9 can comprise any of a variety of structures, including polymeric monofilament materials, braided or woven materials, metal ribbon or wire, or conventional guidewires as are well known in the art. In one embodiment, the second sheath comprises a thin walled PTFE extrusion.

As will be explained in more detail below, in use, when the outer sheath 34 is proximally retracted, the constrained contralateral branch 56 will be exposed. The constrained contralateral branch can then be positioned in the contralateral iliac and deployed by proximally retracting the contralateral guidewire 9 through a second percutaneous puncture site using any of a variety of techniques known to those of skill in the art. One such technique is disclosed in U.S. Pat. No. 6,660,030, entitled “Bifurcation graft deployment catheter,” filed Dec. 20, 2000, the disclosure of which is incorporated in its entirety herein by reference. Proximally withdrawing the contralateral guidewire 9 proximally withdraws the second sheath 48 from the contralateral branch graft portion 56. The contralateral branch graft portion 56 thereafter self expands to fit within the contralateral iliac. In one embodiment, the graft is seated against the bifurcation by proximally withdrawing the contralateral guidewire 9 and the delivery system. This pulls the two ends of the graft in a proximal direction until the bifurcation of the graft is seated against the bifurcation of the vascular system.

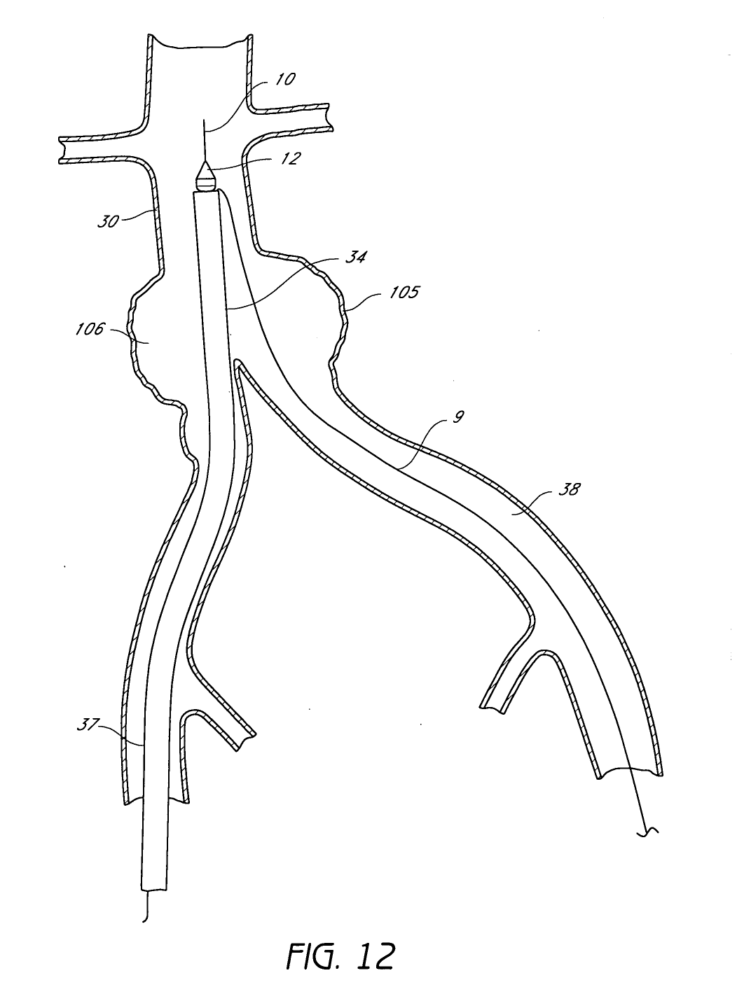

With reference now to FIG. 12, the delivery system 100 described for deploying a multi-segmented endoluminal vascular prosthesis is depicted in situ in the abdominal aorta 30. The delivery system 100 can be percutaneously (or surgically) inserted into an access site, such as a femoral artery puncture (not shown). The delivery system 100 can then be advanced along a guidewire 10 through the ipsilateral iliac 37 and into the abdominal aorta 30. As mentioned above, in modified embodiments, the delivery system can be advanced into other portions of a patient's anatomy. For example, the system 100 can be advanced into the aortic arch such that the vascular graft can be deployed in the aortic arch and/or one or more of the carotid arteries or other branch vessels. In certain embodiments, a contralateral guidewire 9 can first be advanced through the ipsilateral iliac 37 and crossed over into the contralateral iliac 38 in accordance with crossover techniques which are well known in the art. See e.g., U.S. Pat. No. 6,440,161, the entirety of which is hereby incorporated by reference herein. The contralateral guidewire 9 can then be advanced distally down the contralateral iliac 38 where it exits the body at a second access site. The contralateral guidewire 9 can then be used to position and deploy a contralateral branch portion of the endoluminal vascular prosthesis. In modified embodiments, the contralateral guidewire 9 and second sheath 48 can be configured for single puncture access as described in U.S. Pat. No. 6,261,316 and U.S. Patent Publication 2004/0167618, the entire contents of these applications being hereby incorporated by reference herein.

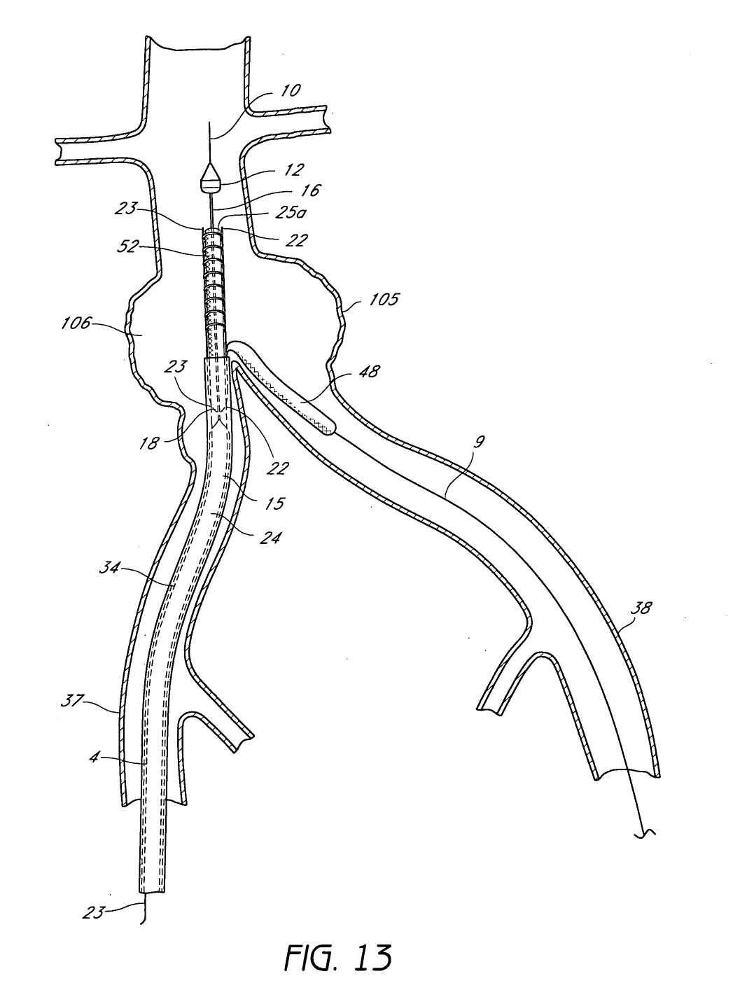

As depicted in FIG. 13, once the delivery system 100 is positioned in the abdominal aorta 30 spanning the aneurysms 105, and 106, the outer sheath 34 may be proximally retracted to expose the contralateral graft portion 56 and the main graft portion 52, which both remain a compressed state. The contralateral graft portion 56 can be maintained in a compressed state by a restraining system, such as the sheath 48 (described above) connected to the proximal end of the contralateral guidewire 9 depicted herein, or any other suitable restraining mechanism. Examples of other suitable mechanisms for constraining the contralateral graft portion which may be used with the present invention, are disclosed in U.S. Pat. No. 6,261,316, entitled “Single puncture bifurcation graft deployment system,” filed Mar. 11, 1999, the disclosure of which is incorporated in its entirety herein by reference.

The main graft portion 52 can be positioned over the distal region 16 of the central core 24 and can be maintained in the compressed state in one embodiment by the series of loops 25a-b attached to the restraining wire 22 extending from the proximal region 15 of the central core 24. As explained above, the loops 25a-b can be wrapped around the compressed main graft portion 52 and held together by the release wire 23 extending from the proximal region 15 of the central core 24 and running through each pair of loops 25a-b along the opposite of the main graft portion 52. The proximal end of the restraining wire 22 can be fixedly attached to the proximal end of the central core 24 while the release wire 23 can extend proximally through the proximal end of the central core 24, so that the release wire 23 may be retracted proximally relative to the fixed restraining wire 22. Alternatively, the proximal end of the restraining wire 22 may also extend proximally through the proximal end of the central core 24 so that the restraining wire 22 and loops 25a-b may be retracted into the outer sheath prior to retracting the delivery system through the patient's blood vessel

The ipsilateral branch portion 54, in turn, can remain constrained within the sheath 18.

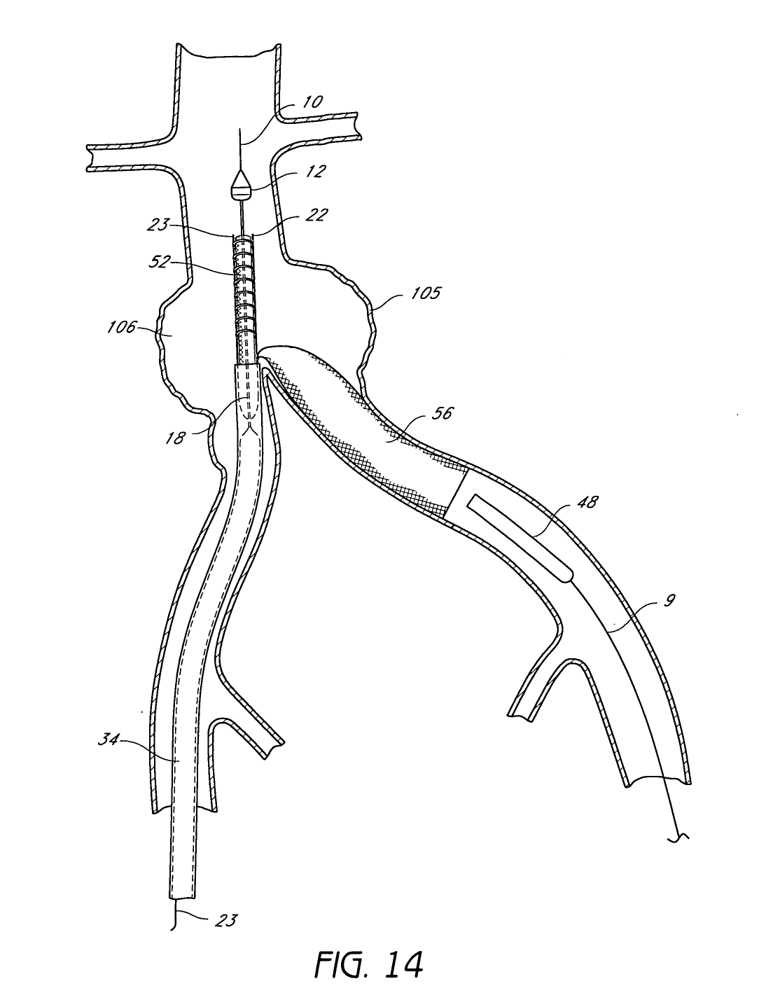

The compressed contralateral graft portion 56 can be positioned in the contralateral iliac 38 by proximally retracting the contralateral guidewire 9 through the second percutaneous access site. As mentioned above, in one embodiment, the graft can seated against the bifurcation by proximally withdrawing the contralateral guidewire 9 and the delivery system 100. This pulls the two ends of the graft in a proximal direction until the bifurcation of the graft is seated against the bifurcation of the vascular system (see FIG. 14). To aid the seating of the graft against the bifurcation, the outer sheath 34 can be advanced until its distal end substantially covers the ipsilateral branch portion 54 and is positioned generally next to the bifurcation between the ipsilateral and contralateral branch portions 54, 56 (see FIG. 14).

As shown in FIG. 14, once the contralateral graft portion 56 is positioned in the contralateral iliac 38, the contralateral graft portion can be deployed, for example by proximally withdrawing the contralateral guide wire 9 to proximally retract the peelable sheath 48 constraining the contralateral graft portion 56. As mentioned above, in certain embodiments, this may be preceded by the step of distally advancing the outer sheath 34 to dock against the bifurcation in order to provide support while the sheath is removed. The contralateral guidewire 9 and sheath 48 can thereafter be proximally withdrawn and removed from the patient, by way of the second percutaneous access site.

In a modified embodiment, the contralateral graft portion 56 can be deployed after the main graft portion 52 is deployed as described below.

As mentioned above, the main graft portion 52 can be released by proximal retraction of the release wire 23 through the proximal region 15 of the central core 24. As shown in FIG. 3, proximal retraction of the release wire 23 can release the pairs of loops 25a-b restraining the main graft portion 52, starting with the distal pair of loops and thus advantageously deploys the main graft portion 52 from the distal end first. In modified embodiments, as described above, the release wire 23 can be configured to deploy the main graft portion 52 from a proximal or middle section first. In another modified embodiment, the release wire 23 can be configured to deploy the main graft portion 52 in sections. The distal end of the outer sheath 34 can abut against the bifurcation of the graft to apply a distal force to the graft as the release wire 23 is withdrawn proximally.

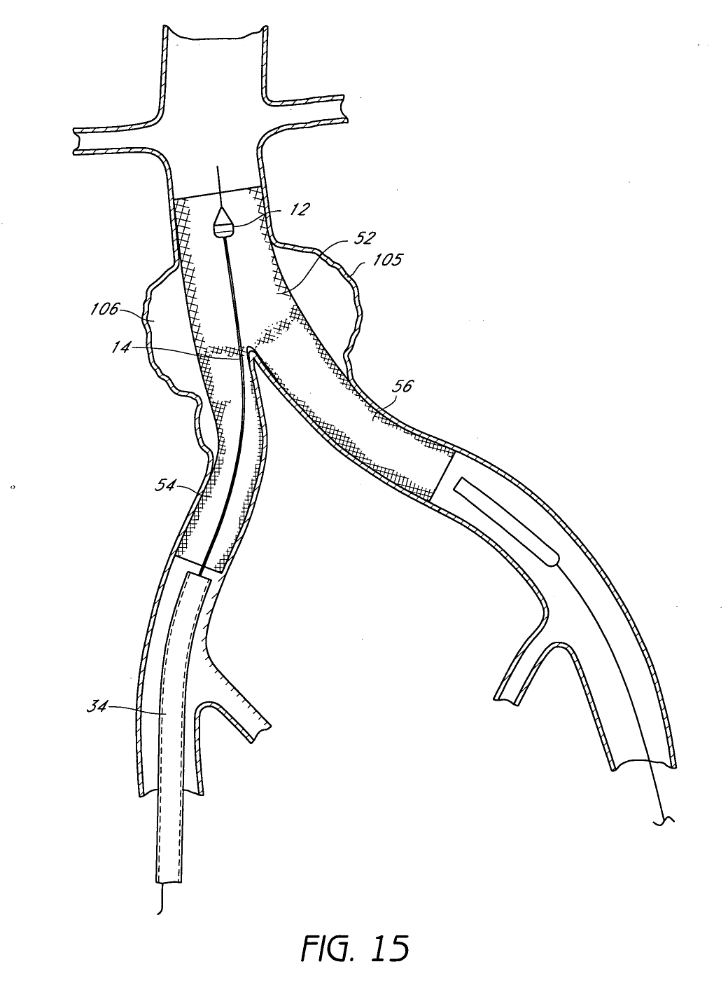

After deployment of the main graft portion 52, the ipsilateral graft portion 54 of the bifurcated stent can still remain constrained within the sheath 18 mounted to the central core 24. As shown in FIG. 15, proximal retraction of the distal region 16 of the central core 24 through the expanded main graft portion 52 will also retract the sheath 18 mounted to the central core 24 and thus deploy the ipsilateral branch 54. Withdrawing the central core 24 will also withdraw the restraining wire 22 into the outer sheath 34.

Once the ipsilateral branch 54 has been deployed, the distal region 16 of the central core 24 and distal cap 12 can be withdrawn through the lumen of expanded ipsilateral graft portion 54 until the distal cap 12 is secured in the aperture at the distal end of the outer sheath 34. The delivery system 100 may then be withdrawn from the patient's vasculature.

As has been mentioned above, while the delivery system is described with respect to deploying a bifurcated stent in the abdominal aortic, it is further envisioned that the delivery system could be used to deliver prosthesis having a main portion and at least one branch portion, or alternatively a prosthesis having only a straight, main graft portion, to other branched intravascular vessels (e.g., the thoracic aorta and a cardiac artery).

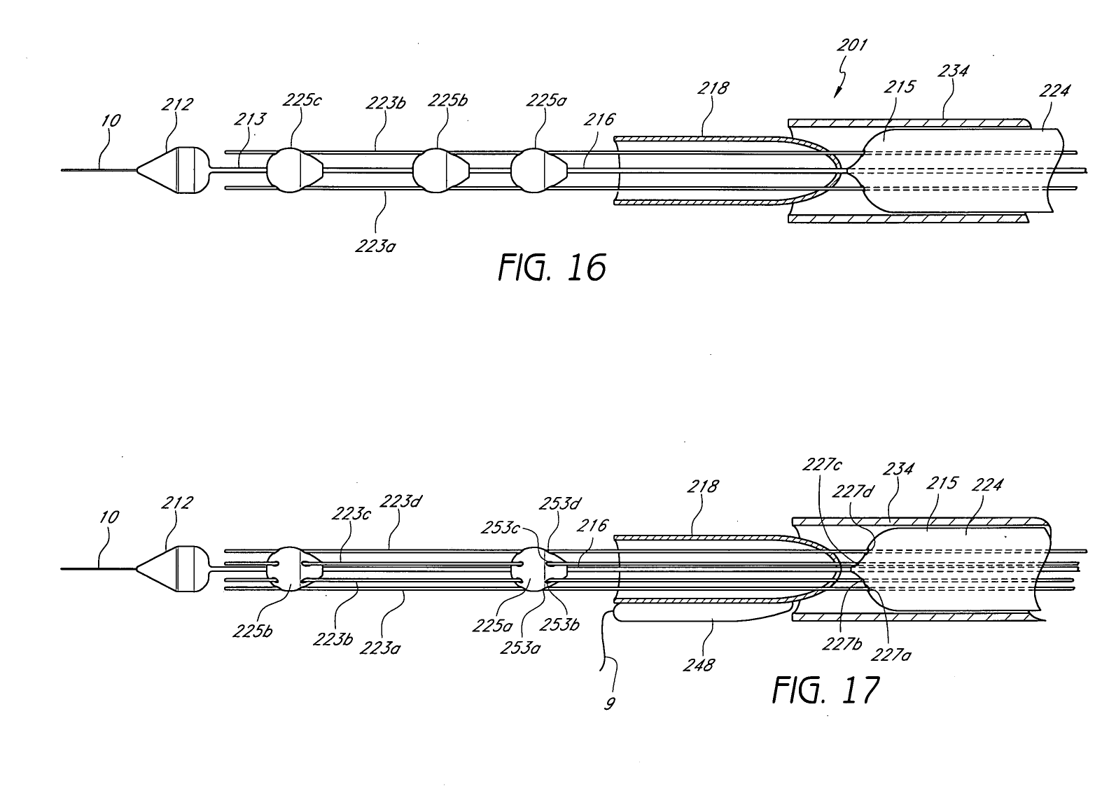

FIGS. 16-17, depict a modified embodiment of a multi-segment delivery system 200. As with the previous embodiment, the delivery system 200 can comprise an elongate flexible multi-component tubular body 201 that includes a central core 224, having a distal region 216 and a proximal region 215, and an outer sheath 234. As described above, in certain embodiments, the central core 224 is preferably axially movably positioned within but rotationally locked to the to the outer sheath 234.

As with the previous embodiments, the tubular outer sheath 234 can be configured to completely cover the main branch portion 52 and both the contralateral and ipsilateral branch portions 54, 56 of the graft 50 and connect with a distal cap 212 mounted on the distal end 213 of the central core 224 in order to provide a smooth, continuous exterior for advancing the delivery system through the patients vasculature.

In certain embodiments, the central core 224 may have a varying cross-sectional diameter such that a proximal region 215 of the central core 224 has a cross-sectional diameter sized to be slidably insertable in the lumen of the outer sheath 234 which necks down to a smaller diameter distal region 216. The distal region 216 the central core 224 is preferably a thin-walled tube designed to track over a guidewire 10, such as a standard 0.035 guidewire. The distal region 216 of the central core 224 preferably has as small an outside diameter as possible to minimize the over all outside diameter of the delivery system, while still providing sufficient column strength to support a compressed vascular stent housed on thereon. The proximal region 215 of the central core 224 can comprise a polyethelene or PTFE extrusion having one or more lumens extending axially through the tubular body. A restraint system configured to internally constrain the main graft portion of a multi-segmented graft can be coupled to the distal region 216 central core 224.

As shown in FIGS. 16-17, the restraint system in this embodiment can include at least two restraint members 225a-b spaced apart on the distal end 216 of the central core 224. Each restraint member 225 can have a plurality of holes 253a-d spaced apart around the circumference of the restraint members 225 and extending axially through the restraint members 225. A plurality of release wires 223a-d can extend distally from a plurality of lumens 227a-d extending axially through the proximal region 215 of the central core 224, as shown in FIGS. 16-17, or alternatively, the release wires may extend in the annular space between the central core 224 and the outer sheath 234. In certain embodiments, as discussed above with respect to FIG. 11, the proximal region 215 of the central core 224 may likewise include a plurality of grooves spaced apart along the circumference and extending longitudinally for creating annular space between the central core 224 and the outer sheath 24 through which the release wires 223a-d may extend. The wires 223 a-d may be operably sized to extend freely from the proximal end of the central core 224 while having a sufficient length to extend distally through the corresponding holes 253a-d on each restraint members 225. In use, the proximal end of the release wires may be pulled proximally to retract the release wires 223a-d from the restraint members 225a-b and thereby release a constrained graft portion.

A vascular graft for use with this embodiment preferably comprises a polymeric sheath covering an wire endoskeleton, as described above. Those skilled in the art will under stand that vascular grafts may have a wire support comprising an endoskeleton situated inside a polymeric sheath or alternatively may comprise an exoskeleton surrounding the polymeric sheath. A vascular stent for use with this embodiment, preferably comprises an endoskeleton such that the release wires 223a-d may be alternately threaded through the links of the endoskeleton and the holes 253a-d of the restraint members 225 located on central core 214 to internally constrict the vascular graft. However, it is envisioned that in alternative embodiments for use with a vascular graft having an exoskeleton, the release wires 223a-d may be threaded through hooks, barbs, and/or holes provided along the inner diameter of the polymeric sheath of the main graft portion.

In use, a vascular graft comprising at least a main graft portion and one branch graft portion can be slidably positioned over the distal region 216 of the central core 224 and positioned so that the main graft portion is located adjacent the restraint members 225a-b. The release wires 223a-d can then be progressively pushed through the restraint members 225 and then threaded in between the links of the wire endoskeleton of the main graft portion. In this manner, the wires 223a-d can hold the main graft portion in a compressed configuration even when the outer sheath 234 is withdrawn. In other embodiments, the wires 225 can be threaded through hooks, barbs, and/or holes provided on the main graft portion.

In certain embodiments, as depicted in FIG. 17, two restraint members 225a-b can be mounted on the distal region 216 of the central core 224 such that they are positioned at the distal and proximal ends of the main graft portion. The release wires 223a-d can be pushed through the holes 253a-d in the restraint members 225a and then woven through the wire links (or other linking mechanism) of the endoskeleton, between the wire endoskeleton and the PTFE sleeve of the vascular graft. Once the main graft portion has been threaded, the release wires 223a-d can be pushed through the holes 253a-d on the distal restraint member 225b to compress the main graft portion. The main graft portion will be constrained internally by the force of the release wires 223a-d on the wire endoskeleton of the graft pulling the graft inward toward the central core 224. When the release wires 223a-d are proximally retracted through the restraint members 225a-b and the links of the wire endoskeleton, the main graft portion will be released and may expand to its unconstrained diameter. Here, the main graft portion will be released starting with the distal end first.

In a modified embodiment, as depicted in FIG. 16, the restraining system may include the two restraint members positioned at the proximal and distal ends of the main graft portion as well as one or more restraint members spaced apart along the mid section of the main graft portion. Here, each of the release wires 223a-d can be progressively threaded through one or more of the restraint members 225a-c and the links of the wire endoskeleton. In addition, certain wires may also be threaded through one or more mid-section restraint members. In use, the release wires can be individually proximally retracted though the restraint members 225 and the wire links of the main graft such that the main graft may be released from the mid section first or alternatively from the proximal end first. For example, in certain embodiments, a fraction of the release wires 223a-d may be threaded through only the midsection restraint member 225b and the restraint member restraint member 225c and the wire links between those restraint members 225b-c. The remainder of the release wires 223a-d can only be threaded through the proximal restraint member 225a and the midsection restraint member 225b and the wire links therebetween. Thus, if the release wires threaded through the proximal restraint member 225a and the mid-section restraint member 225b are proximally retracted before the release wires threaded through the distal restraint member 225c and the mid-section restraint member 225b, the main graft portion will be released starting with the proximal end first.

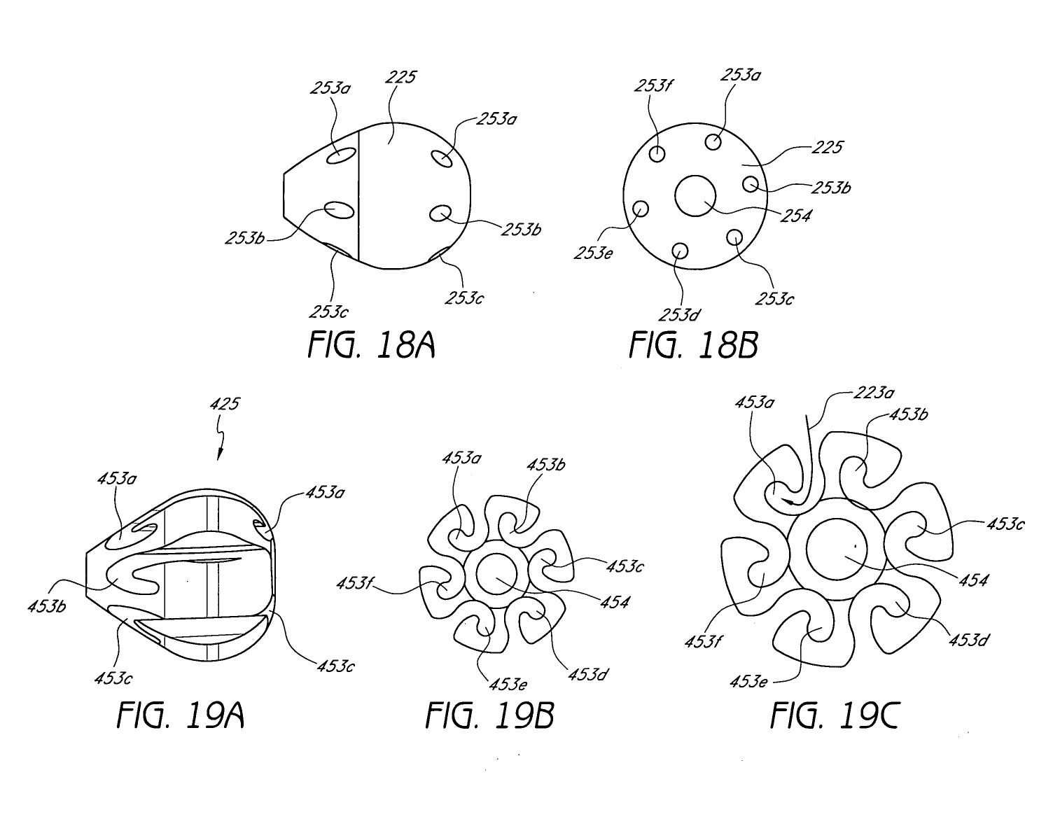

The restraint members can be manufactured any suitable material such as a plastic polymer or a metal, and may have an outer cross-sectional diameter of between about 0.180″-0.2″, alternatively about 0.195.″ In certain embodiments, as depicted in FIGS. 18A-B, the restraint members may have six holes 253a-f extending around the circumference of the restraint member 225, however, it is envisioned that the restraint members 225a-c may have any more or fewer holes 253 as needed to constrain the main graft portion. For example, the restraint members 225 a-c can have between two to ten holes, alternatively between four to eight holes, alternatively 6 holes. The number of restraining wires 223 extending from the proximal region 215 of the central core 224 will preferably correspond to the number of wire links extending circumferentially about the endoskeleton of the vascular graft such that each wire link is pulled inward towards the central core 224 by a restraining wire, thus providing maximum possible compression of the main graft portion. The number of holes 253 in the restraint members 225 preferably corresponds to the number of restraining wires 223 provided.

In a modified embodiment, as depicted in FIGS. 19A-C, the restraint members 425 can comprise one or more slots or grooves 453a-f through which the restraining wires 223 may be snapped in order to connect the wires to the restraint members. Here, instead of progressively threading the release wires 223 through the restraint members 425 and the wire links of the stent, the release wires 223 can be threaded through the entire length of the main graft portion and then the release wires 223 may be pushed into the grooves of the restraint members 225. The main graft portion will be constrained internally by the force of the release wires 223a-d on the wire stent of the graft pulling the main graft portion inward toward the distal region 216 of the central core 224. As shown in FIG. 19C, the grooves 453a-f can be curved such that once the wires have been snapped into the groove 453, the outward force of the main graft portion pulling on the release wire 223 will force the release wires 223 further into interior portion of the groove 453 and will keep the restraint member 425 from moving axially along the length of the distal region 216 of the central core 224. In use, the release wires 223 can be proximally retraced through the wire links of the main graft portion and the grooves 453 of the restraint members. When the release wires 223a-d are proximally retracted through the restraint members 425 and the links of the wire stent, the main graft portion will be released and may expand to its unconstrained diameter. The distal end of the outer sheath 234 can abut against the bifurcation of the graft to apply a distal force to the graft as the release wires 223 are withdrawn proximally.

As shown in FIGS. 16-17, the distal region 216 of the central core 224 can further comprise a tubular sheath 218 mounted proximal to the restraint members 225 for externally constraining the ipsilateral branch portion of the graft. In one embodiment, the sheath 218 comprises a thin walled PTFE extrusion having an outer diameter of about 0.215″ and an axial length of about 7.5 cm. The distal end of the sheath 218 can be open-ended to allow the ipsilateral branch to be connected to the main graft portion, while the proximal end of the sheath 218 can be necked down such as by heat shrinking to secure the tubular sheath 218 to the distal region 216 of the central core 224. Proximal retraction of the central core 224 will in turn proximally retract the sheath 218, thereby deploying the ipsilateral branch graft portion as described above. In use, once the main graft portion has been deployed by proximal traction of the release wires 223 through the restraint members 225 mounted on the central core 224, the central core 224 will be proximally retracted to deploy the ipsilateral branch graft portion

In certain embodiments, a second, contralateral graft portion, may also extend proximally from the main graft portion. In certain embodiments, the contralateral branch portion of the vascular graft may be held in a compressed configuration by a second sheath 248. As described above, the second sheath 248 may be positioned alongside the constrained ipsilateral branch portion in the annular space between the tubular sheath 218 containing the ipsilateral branch portion and the outer sheath 234 of the delivery system. In use, when the outer sheath 234 is proximally retracted, the compressed contralateral branch will be exposed. The compressed contralateral branch may then be positioned in the contralateral iliac and deployed, for example, by proximal retraction of a contralateral guidewire connected to the second sheath. In certain embodiments, the contralateral branch portion may be deployed prior to or after proximally retracting the release wires 223a-f to deploy the main graft portion. This may be advantageous in that the correct position for the branch graft portion may be determined prior to deploying the main graft portion. In addition, the expanded branch graft portion can act as an anchor to stabilize the main graft portion as it is deployed and help to prevent trauma to the blood vessel walls. However, as mentioned above, it is envisioned that the contralateral branch can be deployed before or after the main graft portion.

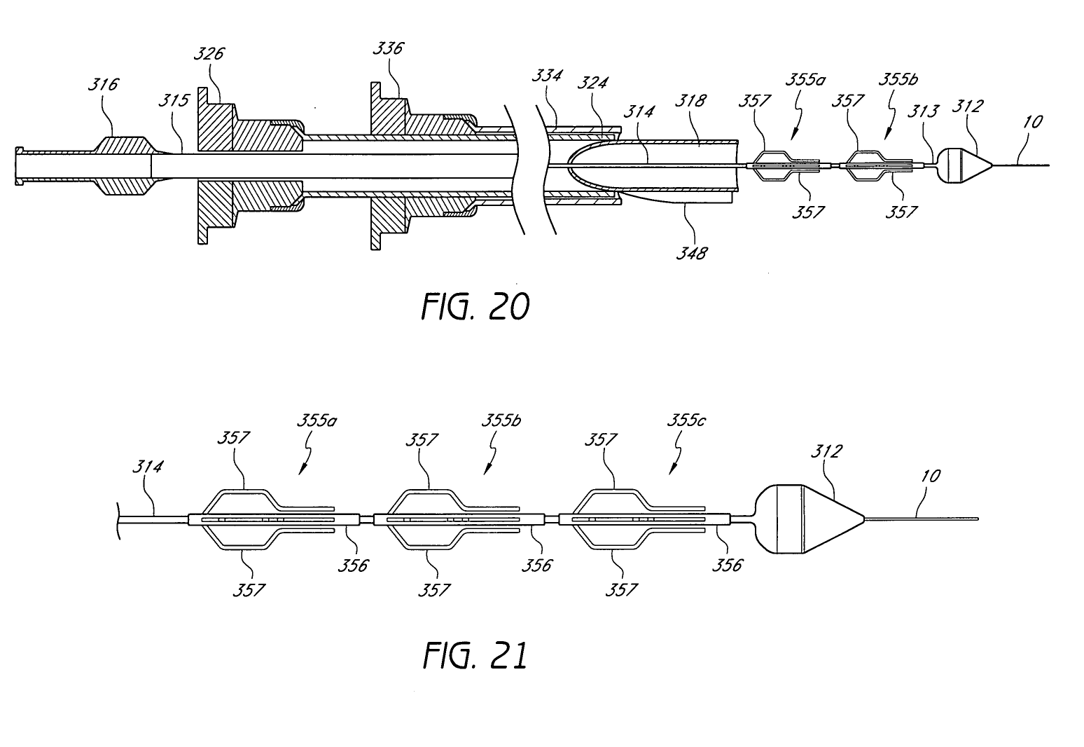

FIGS. 20-21 depict another modified embodiment of a multi-segment delivery system 300. As with the previous embodiments, the delivery system 300 can comprise an elongate flexible multi-component tubular body 301 that includes a central core 314, a intermediate member 324 and an outer sheath 334. The central core 314 can be axially movably positioned within but rotationally locked to the intermediate member 324 which is axially movably positioned within but rotationally locked to the outer sheath 334.

As with the previous embodiments, the tubular outer sheath 334 can be configured to completely cover the main branch portion and both the contralateral and ipsilateral branch portions of the graft and connect with a distal cap 312 mounted on the distal end 313 of the central core 314 in order to provide a smooth, continuous exterior for advancing the delivery system through the patients vasculature.

The central core can be a thin-walled tube having a central core lumen designed to track over a guidewire 10, such as a standard 0.035 guidewire. The central core 314 preferably has as small an outside diameter as possible to minimize the over all outside diameter of the delivery system, while still providing sufficient column strength to support a compressed vascular stent housed on the distal end thereof. In this embodiment, the system can include a plurality of curved hooks or barbs 357 that can be mounted along the distal end 313 of the central core 314. The hooks 357 can be configured into groups of two or more hooks that extend around the circumference of the central core 314. Each grouping of hooks 355a-b can be axially spaced along the longitudinal axis of the central core 314. As depicted, in FIG. 20, the central core 314 can have a proximal group of hooks 355a and a distal group of hooks 355b spaced apart such that the proximal group of hooks 355a can engage the proximal end of the main graft portion while the distal group of hooks 355b can engage the distal end of the main graft portion. However, in modified embodiments, the central core 314 can have two, alternatively three, alternatively four groups of hooks 355 spaced apart along its longitudinal axis depending upon the length of the main graft portion which the hooks 355 will constrain.

In certain embodiments as depicted in FIG. 21, each group of hooks 355a-b can be mounted on a segment of a hypotube 356 operably sized to be axially positioned over the central core 314. To prevent longitudinal movement of the hypotube segments 356 along the central core 314, the hypotube segments 356 can be friction fit or bonded to the central core 314 using epoxy, heat or any other suitable method known to those skilled in the art.

The hooks 357 can be mounted to the hypotube segments on the proximal end of the hook 357 and have a distal opening in order to grasp and engage the wire links of wire stent of the main graft portion. The hooks 357 are configured to grasp the internal wire links of the main graft portion and pull them toward the central core 314, thereby compressing the main graft portion against the central core 314. In certain embodiments, as depicted in FIGS. 20-21, the hooks 357 are curved in a proximal region and flatten out and extend parallel to the longitudinal axis near the opening of the hook. The curvature in the hooks can facilitate loading of the wire links onto the hooks and ensures that the deployment force required to release the wire links is not excessive.

In use, a vascular graft may be slidably positioned over the distal end 313 of the central core 314 and positioned so that the main graft portion is located distally of the groups of hooks 355a-b. The wire links (or other linking mechanism, such as, for example, holes, loops, barbs) of the main graft portion may then be loaded on to the hooks 357 by pushing the links down and through the distal opening in the hooks 357. When the central core 314 is proximally retracted with respect to the main graft portion, the wire links will slide out the distal opening of the hooks and be released, thereby expanding the main graft portion to its unconstrained diameter. The distal end of the outer sheath 34 can abut against the bifurcation of the graft to apply a distal force to the graft as the hooks 357 are withdrawn proximally.

The intermediate member 324 can comprise an elongate tubular body having a central core lumen adapted to axially slidably track over the central core 314. A tubular sheath 318 can be secured to the distal end of the intermediate member 324 for externally constraining the ipsilateral branch portion of the graft as described above. In one embodiment, the sheath 318 comprises a thin walled PTFE extrusion having an outer diameter of about 0.215″ and an axial length of about 7.5 cm. The distal end of the sheath 318 can be open-ended to allow the ipsilateral branch to be connected to the main graft portion, while the proximal end of the sheath 318 is necked down such as by heat shrinking to secure the tubular sheath 318 to the intermediate member 324. Proximal retraction of the intermediate member 324 will in turn proximally retract the sheath 318, thereby deploying the ipsilateral branch graft portion. In use, once the main graft portion has been deployed by proximal traction of the central core 314, the intermediate member 324 can be proximally retracted to deploy the ipsilateral branch graft portion. Once the ipsilateral branch graft portion has been deployed, the central core 314 and distal tip 312 can be further proximally retracted though the central core lumen of the expanded ipsilateral branch graft portion into the outer sheath 334 and the delivery system 300 can be completely withdrawn form the patient.

In certain embodiments, a second, contralateral graft portion may also extend proximally from the main graft portion. For example, the contralateral branch portion of the vascular graft can be held in a compressed configuration by a second sheath 348 attached to a contralateral guidewire, or any other suitable external compression means known to those skilled in the arts. The compressed contralateral branch can be positioned alongside the constrained ipsilateral branch portion in the annular space between the tubular sheath 318 and the outer sheath 334 of the delivery system. In use, when the outer sheath 334 is proximally retracted, the compressed contralateral branch will be exposed. The compressed contralateral branch can then be positioned in the contralateral iliac and deployed, for example, by proximal retraction on the contralateral guidewire. In certain embodiments, the contralateral branch portion can be deployed prior to proximally retracting the central core 314 to deploy the main graft portion. In other embodiments, the contralateral branch can be deployed before or after the main graft portion.

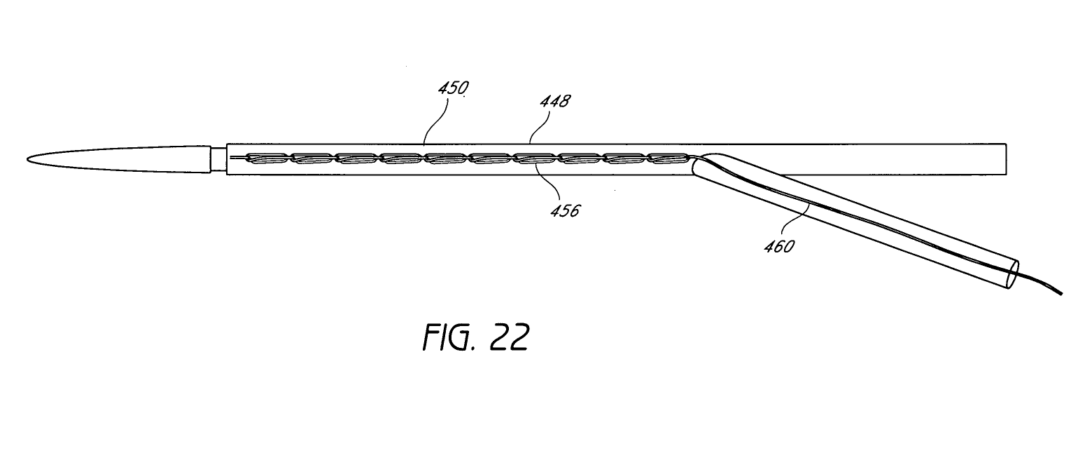

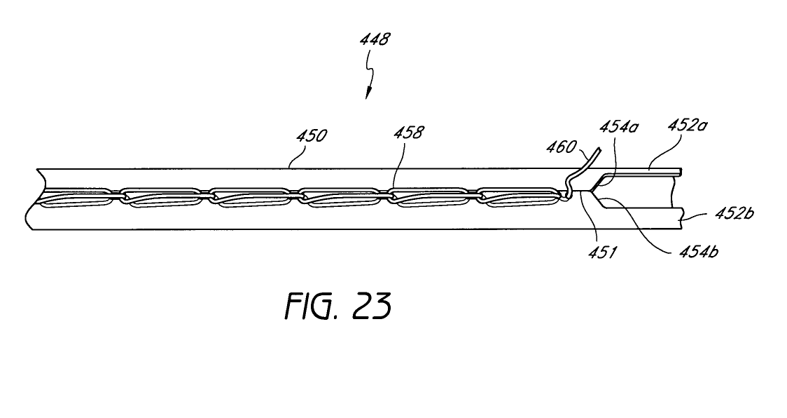

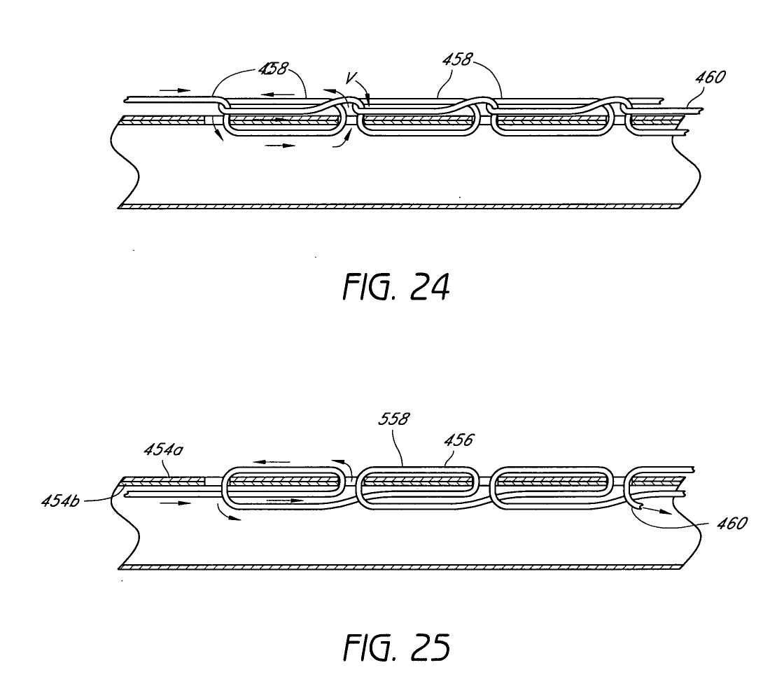

With reference to FIGS. 22-23, an embodiment of a sheath 448 that may be used for compressing the contralateral branch portion is shown. While this embodiment of a sheath is shown with reference to compressing a main branch graft portion, it is envisioned that the sheath could alternatively be used to compress and deliver other portions of a multi-segmented vascular graft, such as a branch graft portion, the entire multi-segmented graft, or a single-segment, straight vascular graft. The sheath 448 is a thin walled, flexible tubular member 450 comprised of a biocompatible material such as PTFE. The tubular member comprises two semicircular sidewalls 452a-b which are attached, or continuous, along one adjacent longitudinal edge 453a-b and remain disconnected along the opposite adjacent longitudinal edge 454a-b, such that the tubular member 450 has a longitudinal slit 451 extending the entire length of the tubular member 450. This permits the tubular member 450 to be opened along its longitudinal axis to release a constrained graft segment. A vascular graft portion may be positioned through the slit 451 in the interior of the tubular member 450. Once the graft has been positioned in the interior cavity of the tubular member, the side walls 452 of the tubular member may be compressed such that the longitudinal edges 454a-b are brought together or overlapped. A suture 456 may then be threaded through both longitudinal edges 454a-b of the sidewalls 452a-b along the entire length of the tubular member 450 to attach the longitudinal edges 454a-b and compress the graft portion in the tubular member 450.

As shown in FIGS. 24-25, the suture 456 may be threaded through the opposing edges 454a-b of the side walls starting at the proximal end of the tubular member using a temporary stitch. The distal end of the suture extends freely from the tubular member and may be pulled distally to open the longitudinal slit 451 in the tubular member 450 from the distal end and thereby release the compressed graft portion. For example, in FIG. 24, each stitch 458 is looped through the previous stitch 458 such that when the distal, free end of the suture is pulled distally, the stitches will be sequentially released. Alternatively, as shown in FIG. 25, the opposing edges 454a-b of the tubular member 450 may be temporarily held together by a looped basting stitch 558 threaded through both opposing edges. In use, once the delivery sheath 448 has been properly positioned, the distal suture end 460 may be pulled proximally to release the stitches 458 and thereby allow the tubular member 450 to open along the longitudinal slit and release the constrained graft portion.

Although the foregoing description of the preferred embodiments of the present invention has shown, described and pointed out the fundamental novel features of the invention, it will be understood that various omissions, substitutions, and changes in the form of the detail of the apparatus as illustrated as well as the uses thereof, may be made by those skilled in the art, without departing from the spirit of the invention.

What is claimed is:

1. A deployment catheter for deploying endoluminal vascular prosthesis having at least a main graft portion, and a first branch graft portion, comprising:
an elongate, flexible catheter body having a proximal end and a distal end and comprising an outer sheath and an inner core that is axially moveable with respect to the outer sheath;
a main graft restraint comprising a main graft release mechanism comprising a plurality of axially spaced restraint members; and
a branch graft restraint comprising a branch graft release mechanism.

2. The deployment catheter of claim 1, wherein the plurality of axially spaced restraint members are positioned within the main graft.

3. The deployment catheter of claim 2, wherein the plurality of axially spaced restraint members are positioned on the central core.

4. The deployment catheter of claim 3, wherein the plurality of axially spaced restraint members are configured such that proximal retraction of the central core with respect to the main graft decouples the plurality of axially spaced restraint members from the main graft.

5. The deployment catheter of claim 2, further comprising a plurality of release wires releasably coupled to the main graft and the plurality of axially spaced restraint members.

6. The deployment catheter of claim 5, wherein each of the restraint members comprise a plurality of openings and the release wires are configured to extend through the plurality of openings.

7. The deployment catheter of claim 5, wherein each of the restraint members comprise a plurality of grooves circumferentially spaced and extending axially along the restraint members; and wherein the release wires are configured to engage the plurality of grooves on the plurality of restraint members.

8. The deployment catheter of claim 1, wherein the plurality of axially spaced restraint members configured to extend around the main graft.

9. The deployment catheter of claim 8, further comprising a release mechanism operably coupled to the plurality of restraint members and wherein the release mechanism is operably coupled to an intermediate member positioned between the inner core and the outer sheath such that proximal retraction of the intermediate member decouples the plurality of restraint members from the release mechanism.

10. The deployment catheter of claim 8, wherein the plurality of axially spaced restraint comprises a plurality of opposing connectors spaced apart along the distal end of the restraining wire and a release wire is configured to extend through the plurality of pairs of opposing connectors to join the opposing connectors.

11. The deployment catheter of claim 10, wherein proximal retraction of the release wire releases the plurality of opposing connectors.

12. The deployment catheter of claim 10, wherein the opposing connectors comprise pairs of wire loops transversely extending from the restraining wire.

13. The deployment catheter of claim 10, wherein the opposing connectors comprise flexible, discontinuous rings having eyelets attached to each end

14. The deployment catheter of claim 1, wherein the branch graft release mechanism comprises an external release mechanism.

15. The deployment catheter of claim 14, wherein the external release mechanism comprises a sheath.

16. The deployment catheter of claim 1, further comprising an intermediate member moveably positioned between the inner core an the outer sheath, the intermediate member a tubular sheath positioned on the distal end for constraining a second branch graft portion.

17. The deployment catheter of claim 16, wherein proximal motion of the intermediate member releases the second branch graft portion.

18. A deployment catheter for deploying a an endoluminal vascular prosthesis stent having a main graft portion, and a first branch graft portion, comprising:
a flexible outer tubular member, having a proximal end and a distal end;
an intermediate tubular member slidably engaged with the outer tubular member, having a proximal end and a distal end;
a central core slidably engaged with the intermediate tubular member, having a proximal end and a distal end, a flexible, conical tip mounted on the distal end
a main graft restraint operatively engaged with the intermediate tubular member and the central core, configured such that proximal retraction of the intermediate tubular member relative to the central core will release the main graft restraint;
wherein proximal retraction of the outer tubular member will release the first compressed branch graft portion.

19. The deployment catheter of claim 18, wherein the main graft restraint comprises an restraint mechanism positioned within the main graft.

20. The deployment catheter of claim 19, wherein the restraint mechanism comprises:
one or more members mounted on the central core, the members having a plurality of openings extending axially through the restraint members; and
a plurality of wires, having proximal ends and distal ends, configured to extend through the plurality of openings on the restraint members, the proximal ends of the wires connected to the intermediate tubular member.

21. The deployment catheter of claim 20, wherein the plurality of wires are configured to be woven through the endoskeleton of the main graft portion and the openings in the restraint members to hold the main graft portion in a compressed state.

22. The deployment catheter of claim 21, wherein proximal retraction of the intermediate tubular member retracts the plurality of wires and releases the main graft portion.

23. The deployment catheter of claim 19, wherein the restraint mechanism comprises:
one or more restraint members coupled to the central core, the restraint members having a plurality of grooves extending axially along the restraint members; and
a plurality of wires coupled configured to extend through the plurality of grooves on the restraint members.

24. The deployment catheter of claim 18, wherein the central core further comprises a tubular sheath configured to constrain a second branch portion in a compressed state; and wherein proximal retraction of the central core releases the second branch graft portion.

25. The deployment catheter of claim 18, wherein the main graft restraint comprises:
a restraint wire having proximal and distal ends, the proximal end connected to the proximal end of the intermediate tubular member;
a plurality of opposing connectors spaced apart along a distal of the restraint wire
a release wire having proximal and distal ends, the distal end of the release wire is configured to extended through the plurality of opposing connectors and connect the plurality of opposing connectors, the proximal end of the release wire configured to extend from the proximal end of the intermediate tubular member; and
wherein proximal retraction of the release wire releases the opposing connectors.

26. The deployment catheter of claim 25, wherein the opposing connectors comprise pairs of wire loops and wherein the release wire is configured to be extended through the pairs of loops.

27. The deployment catheter of claim 25, wherein the opposing connectors are configured to surround a main graft portion disposed on the central core and to constrain the main graft portion in a compressed state when the release wire extends through the opposing connectors.

28. The deployment catheter of claim 27, wherein proximal retraction of the release wire releases the main graft portion.

29. The deployment catheter of claim 25, wherein the intermediate tubular member comprises a multi-lumen tubular member having a central core lumen, and two wire lumens; wherein the restraint wire and the release wires extend through the wire lumens.

30. The deployment catheter of claim 25, wherein the intermediate tubular member comprises a tubular member having two grooves extending along the length of the inner tubular member and wherein the restraint wire and the release wires are slidably insertable in the grooves.

31. A deployment catheter for deploying an endoluminal vascular prosthesis having a main graft portion, and a first branch portion, comprising
a flexible outer tubular member;
a central core slidably engaged with the outer tubular member;
a main graft portion disposed on the central core and constrained in a compressed state by an internal restraint having a first release mechanism;
a compressed branch graft portion having a second release mechanism, the compressed branch graft portion constrained by the flexible outer tubular member;
wherein the compressed branch graft portion is released by proximal traction of the outer tubular member; and
wherein the first release mechanism is engaged by proximal traction.

32. The deployment catheter of claim 31, wherein the, wherein the internal restraint mechanism comprises a plurality of hooks spaced apart on the central core, the hooks configured to grasp the endoskeleton of the main graft portion.

33. The deployment catheter of claim 32, the hooks further comprise a plurality of distal facing prongs radially disposed on the central core.

34. The deployment catheter of claim 32, wherein proximal motion of the central core releases the links of the main graft portion from the hooks.

35. A method of deploying an endoluminal vascular prosthesis in a patients aortic artery comprising:
advancing a deployment catheter containing an endoluminal vascular prosthesis comprising a compressed main graft portion, a compressed ipsilateral branch portion and a compressed contralateral branch portion, beyond a bifurcation in the aorta;
retracting an outer sheath of the deployment catheter to expose the main graft portion and the contralateral branch portion of the prosthesis;
proximally retracting release member to release a plurality of release mechanisms axially spaced along the main graft to release the main graft portion; and
withdrawing the deployment catheter.

36. The method of claim 35, further complying deploying the contralateral graft portion by removing a peelable sheath from the contralateral graft portion.

37. The method of claim 35, wherein proximally retracting the release member comprising releasing release mechanism positioned within the main graft.

38. The method of claim 35, wherein proximally retracting the release member comprising releasing release mechanism positioned outside the main graft.

39. The method of claim 35, wherein the deployment catheter further comprises a sheath for constraining the ipsilateral branch portion and wherein the proximally retracting the deployment catheter comprises retracting the sheath from the ipsilateral branch portion.
